(19) United States
(12) Patent Application Publication
(10) Pub. No.: US 2022/0205995 A1
(43) Pub. Date: Jun. 30, 2022

(54) DEVICE FOR PERSONAL PREDICTIVE ENRICHMENT OF A BIOMARKER AND METHODS OF USE THEREOF

(71) Applicant: SEQ BIOMARQUE, LLC, Yorba Linda, CA (US)

(72) Inventors: Sean KELLY, Yorba Linda, CA (US); Christopher A. HAMM, Chicago, IL (US)

(21) Appl. No.: 17/606,503

(22) PCT Filed: Apr. 24, 2020
(86) PCT No.: PCT/US2020/029821

§ 371 Date: Oct. 26, 2021

Related U.S. Application Data
(60) Provisional application No. 62/839,165, filed on Apr. 26, 2019.

(51) Int. Cl. G01N 33/543; G01N 33/68

(52) U.S. Cl. CPC G01N 33/54366; G01N 33/6896

(57) ABSTRACT

Methods and devices for enriching a target biomarker from a bodily fluid of a subject are provided. A risk profile for the subject is used to predict that a target biomarker is present in the bodily fluid. The bodily fluid is contacted with a set of immuno-affinity inserts coated with affinity molecules specific for the target biomarker under conditions for the affinity molecules to bind the target biomarker. The affinity molecules bound to the target biomarker are separated from the bodily fluid.

### CROSS REFERENCE TO RELATED APPLICATIONS

The present application claims priority to U.S. Provisional Patent Application Ser. No. 62/839,165, filed Apr. 26, 2019, which is incorporated herein by reference in its entirety.

### TECHNICAL FIELD

The present disclosure relates generally to devices and methods for enriching a biomarker from a bodily fluid sample from a subject.

### BACKGROUND

Cancer is characterized by uncontrolled cell reproduction. It is believed that one-third of all people in the United States will develop cancer. Although remarkable progress has been made in understanding the biological basis of and in treating cancer, cancer remains second only to cardiac disease as the main cause of death in the United States.

The discovery of biomarkers constitutes a significant advancement to the field in cancer diagnosis. Cancer biomarkers are especially useful for early detection or diagnosis of the disease. Biomarkers can be used to screen patients, for classifying the different stages or grades of cancers and to predict prognosis and resistance to therapy. A biomarker marker can be produced by a tumor itself or by the body as a result of the disease. These biomarkers may be produced in small numbers in the cancerous tissues and often secreted to body fluids like blood, serum, urine etc.

The identification of tumor markers suitable for early detection and diagnosis of cancer would improve the clinical outcome of patients, especially those presenting vague or no symptoms. Accordingly, there is a need for improved methods of detection and diagnosis of cancer as well as methods for monitoring the progress of the disease and monitoring the progress of various treatments including point of care or point of use devices capable of quantitating predictive biomarker(s).

### SUMMARY

The present disclosure provides methods for enriching a biomarker (e.g., a target biomarker) from a bodily fluid of a subject. The methods may comprise predicting that a biomarker (e.g., a target biomarker) is present in the bodily fluid; contacting the bodily fluid with one or more immuno-affinity inserts (e.g., particles, tubes and/or filaments) coated with affinity molecules specific for the biomarker under conditions for the affinity molecules to bind the biomarker; and separating the affinity molecules bound to the biomarker from the bodily fluid.

In some embodiments of each or any of the above- or below-mentioned embodiments, the methods further comprise purifying the target biomarker from the affinity molecules.

In some embodiments of each or any of the above- or below-mentioned embodiments, the methods further comprise detecting the target biomarker.

In some embodiments of each or any of the above- or below-mentioned embodiments, the target biomarker is predicted to be present in the bodily fluid based on the risk profile for the subject.

In some embodiments of each or any of the above- or below-mentioned embodiments, the subject's risk profile is based on age, weight, height, ethnicity, medical history, a physical examination, a patient interview, a computerized test result, a medical image, a risk-related public health initiative or directive, or any combination thereof. In a preferred embodiment, the subject's risk profile is based on whether one or more target biomarkers were identified as being present or absence in a bodily fluid from the subject in one or more prior tests and/or the subject's age, weight, height, ethnicity, medical history, a physical examination, an interview, a computerized test result, a medical image, a risk-related public health initiative or directive, etc. For example, a subject's risk profile may take into account that a target biomarker was identified as being present in a bodily fluid from a subject at a first time. Additionally, for example, a subject's risk profile may take into account that a target biomarker was identified as being present in a bodily fluid from a subject at a first time, and a second time, and so on.

In some embodiments of each or any of the above- or below-mentioned embodiments, the subject's risk profile is determined based upon the subject's membership in a class of subjects, wherein all subjects in the class are at risk of a common disease, disorder, or condition.

In some embodiments of each or any of the above- or below-mentioned embodiments, the target biomarker is a protein, a nucleic acid, a peptide, a polymer, an amino acid or a glycan.

In some embodiments of each or any of the above- or below-mentioned embodiments, the bodily fluid is blood, plasma, saliva, tears, urine, amniotic fluid, cerebrospinal fluid, pleural fluid, or peritoneal fluid.

In some embodiments of each or any of the above- or below-mentioned embodiments, the bodily fluid is blood.

In some embodiments of each or any of the above- or below-mentioned embodiments, the immuno-affinity inserts are magnetic.

In some embodiments of each or any of the above- or below-mentioned embodiments, the immuno-affinity inserts are fluorescent.

In some embodiments of each or any of the above- or below-mentioned embodiments, the bodily fluid is contacted with more than one set of immuno-affinity inserts, and wherein each set is coated with a different affinity molecule.

In some embodiments of each or any of the above- or below-mentioned embodiments, the subject is predicted to have a cancer.

In some embodiments of each or any of the above- or below-mentioned embodiments, the cancer is non-melanoma skin cancer, lung cancer, breast cancer, prostate cancer, colorectal cancer, bladder cancer, melanoma, non-Hodgkin lymphoma, ovarian cancer, cervical cancer, or pancreatic cancer.

In some embodiments of each or any of the above- or below-mentioned embodiments, the subject is predicted to have Alzheimer's disease.

In some embodiments of each or any of the above- or below-mentioned embodiments, the target biomarker is tau protein.

In some embodiments of each or any of the above- or below-mentioned embodiments, the tau protein is hyperphosphorylated.

In some embodiments of each or any of the above- or below-mentioned embodiments, the affinity molecules are present in a soluble affinity matrix (SAM).

In some embodiments of each or any of the above- or below-mentioned embodiments, release of the immuno-affinity inserts from the SAM is triggered by pH or osmolality of the bodily fluid.

In some embodiments of each or any of the above- or below-mentioned embodiments, a first set of immuno-affinity inserts is present in a first layer of the SAM.

In some embodiments of each or any of the above- or below-mentioned embodiments, a second set of immuno-affinity inserts is present in a second layer of the SAM.

In some embodiments of each or any of the above- or below-mentioned embodiments, the SAM comprises a first layer that elutes a set of immuno-affinity inserts having affinity molecules that specifically bind one or more undesired biomarkers.

In some embodiments of each or any of the above- or below-mentioned embodiments, the SAM comprises a second layer that elutes a set of immuno-affinity inserts coated with affinity molecules specific for a first target biomarker.

In some embodiments of each or any of the above- or below-mentioned embodiments, the SAM comprises a third layer that elutes a set of immuno-affinity inserts coated with affinity molecules specific for a second target biomarker.

In some embodiments of each or any of the above- or below-mentioned embodiments, a first set of immuno-affinity inserts is released from the SAM at a first predetermined time and a second set of immuno-affinity inserts is released from the SAM at a second predetermined time.

In some embodiments of each or any of the above- or below-mentioned embodiments, the second predetermined time is later than the first predetermined time.

In some embodiments of each or any of the above- or below-mentioned embodiments, the affinity molecules are selected from the group consisting of antibodies, antibody fragments, enzymes, fragments of enzymes, enzyme substrates, fragments of enzyme substrates, nucleotides, oligonucleotides, polynucleotides, receptors, aptamers, nanobodies, fragments of receptors, ligands, fragments of enzymes, other proteins, amino acids, peptides, polypeptides, oligopeptides, saccharides, disaccharides, polysaccharides, glycoproteins, proteoglycans, and natural and synthetic polymers.

In some embodiments of each or any of the above- or below-mentioned embodiments, the immuno-affinity inserts comprise one of particles, tubes and filaments.

The present disclosure also provides methods for enriching a target biomarker from a bodily fluid of a subject comprising obtaining the bodily fluid comprising a target biomarker from the subject; flowing the bodily fluid through a first component containing one or more sets of immuno-affinity inserts coated with affinity molecules specific for the target biomarker, and flowing the bodily fluid through a second component that retain the immuno-affinity inserts bound to the target biomarker.

In some embodiments of each or any of the above- or below-mentioned embodiments, the set of immuno-affinity inserts are magnetic or fluorescent.

In some embodiments of each or any of the above- or below-mentioned embodiments, the first component is a cartridge.

In some embodiments of each or any of the above- or below-mentioned embodiments, the second component is a vial or a cartridge.

In some embodiments of each or any of the above- or below-mentioned embodiments, the target biomarker is associated with a disease, disorder, or condition.

In some embodiments of each or any of the above- or below-mentioned embodiments, the target biomarker is tau protein and the disease is Alzheimer's disease.

In some embodiments of each or any of the above- or below-mentioned embodiments, the method further comprise purifying the target biomarker from the affinity molecules.

In some embodiments of each or any of the above- or below-mentioned embodiments, the methods further comprise detecting the target biomarker.

In some embodiments of each or any of the above- or below-mentioned embodiments, the target biomarker is predicted to be present in the bodily fluid based on the subject's risk profile.

In some embodiments of each or any of the above- or below-mentioned embodiments, the subject's risk profile is based on age, weight, height, ethnicity, medical history, a physical examination, a patient interview, a computerized test result, a medical image, a risk-related public health initiative or directive, or any combination thereof. In a preferred embodiment, the subject's risk profile is based on whether one or more target biomarkers were identified as being present or absence in a bodily fluid from the subject in one or more prior tests and/or the subject's age, weight, height, ethnicity, medical history, a physical examination, an interview, a computerized test result, a medical image, a risk-related public health initiative or directive, etc. For example, a subject's risk profile may take into account that a target biomarker was identified as being present in a bodily fluid from a subject at a first time. Additionally, for example, a subject's risk profile may take into account that a target biomarker was identified as being present in a bodily fluid from a subject at a first time, and a second time, and so on.

In some embodiments of each or any of the above- or below-mentioned embodiments, the subject's risk profile is determined based upon the subject's membership in a class of subjects, wherein all subjects in the class are at risk of a common disease, disorder, or condition.

In some embodiments of each or any of the above- or below-mentioned embodiments, the affinity molecules for a disease, disorder, or condition are selected for use in the device where the subject's risk profile indicates that the likelihood of developing the disease, disorder, or condition is 50% or greater, 55% or greater, 60% or greater, 65% or greater, 70% or greater, 75% or greater, 80% or greater, 85% or greater, 90% or greater, or 95% or greater.

In some embodiments of each or any of the above- or below-mentioned embodiments, the target biomarker is a protein, a nucleic acid, a peptide, a polymer, an amino acid or a glycan.

In some embodiments of each or any of the above- or below-mentioned embodiments, the bodily fluid is blood, plasma, saliva, tears, urine, amniotic fluid, cerebrospinal fluid, pleural fluid, or peritoneal fluid.

In some embodiments of each or any of the above- or below-mentioned embodiments, the bodily fluid is blood.

In some embodiments of each or any of the above- or below-mentioned embodiments, the immuno-affinity inserts comprise one of particles, tubes and filaments.

In some embodiments of each or any of the above- or below-mentioned embodiments, the bodily fluid is contacted with more than one set of immuno-affinity inserts, and wherein each set is coated with a different affinity molecule.

In some embodiments of each or any of the above- or below-mentioned embodiments, the subject is predicted to have a cancer.

In some embodiments of each or any of the above- or below-mentioned embodiments, the cancer is non-melanoma skin cancer, lung cancer, breast cancer, prostate cancer, colorectal cancer, bladder cancer, melanoma, non-Hodgkin lymphoma, ovarian cancer, cervical cancer, or pancreatic cancer.

In some embodiments of each or any of the above- or below-mentioned embodiments, the subject is predicted to have Alzheimer's disease.

In some embodiments of each or any of the above- or below-mentioned embodiments, the target biomarker is tau protein.

In some embodiments of each or any of the above- or below-mentioned embodiments, the tau protein is hyperphosphorylated.

In some embodiments of each or any of the above- or below-mentioned embodiments, the affinity molecules are present in a soluble affinity matrix (SAM).

In some embodiments of each or any of the above- or below-mentioned embodiments, release of the set of immuno-affinity inserts from the SAM is triggered by wetness, pH or osmolality of the bodily fluid.

In some embodiments of each or any of the above- or below-mentioned embodiments, a first set of immuno-affinity inserts is present in a first layer of the SAM.

In some embodiments of each or any of the above- or below-mentioned embodiments, a second set of immuno-affinity inserts is present in a second layer of the SAM.

In some embodiments of each or any of the above- or below-mentioned embodiments, the SAM comprises a first layer that elutes a set of immuno-affinity inserts having affinity molecules that specifically bind one or more undesired biomarkers.

In some embodiments of each or any of the above- or below-mentioned embodiments, the SAM comprises a second layer that elutes the set of immuno-affinity inserts coated with affinity molecules specific for the target biomarker.

In some embodiments of each or any of the above- or below-mentioned embodiments, the first set of immuno-affinity inserts is released from the SAM at a first predetermined time and the second set of immuno-affinity inserts is released from the SAM at a second predetermined time.

In some embodiments of each or any of the above- or below-mentioned embodiments, the second predetermined time is later than the first predetermined time.

In some embodiments of each or any of the above- or below-mentioned embodiments, the affinity molecules are selected from the group consisting of antibodies, antibody fragments, enzymes, fragments of enzymes, enzyme substrates, fragments of enzyme substrates, nucleotides, oligonucleotides, polynucleotides, receptors, aptamers, nanobodies, fragments of receptors, ligands, fragments of enzymes, other proteins, amino acids, peptides, polypeptides, oligopeptides, saccharides, disaccharides, polysaccharides, glycoproteins, proteoglycans, and natural and synthetic polymers.

The present disclosure also provides methods for diagnosing a subject with a disease, disorder, or condition comprising predicting that a target biomarker is present in the bodily fluid; obtaining a bodily fluid from the subject; flowing the bodily fluid through a device containing affinity molecules specific for the target biomarker; and identifying the presence of the target biomarker in the bodily fluid, wherein the subject is diagnosed with the disease, disorder, or condition where the target biomarker is identified in the bodily fluid.

In some embodiments of each or any of the above- or below-mentioned embodiments, the methods further comprise purifying the target biomarker from the affinity molecules.

In some embodiments of each or any of the above- or below-mentioned embodiments, the methods further comprise detecting the target biomarker.

In some embodiments of each or any of the above- or below-mentioned embodiments, the target biomarker is predicted to be present in the bodily fluid based on the subject's risk profile.

In some embodiments of each or any of the above- or below-mentioned embodiments, the subject's risk profile is based on age, weight, height, ethnicity, medical history, a physical examination, a patient interview, a computerized test result, a medical image, a risk-related public health initiative or directive, or any combination thereof. In a preferred embodiment, the subject's risk profile is based on whether one or more target biomarkers were identified as being present or absence in a bodily fluid from the subject in one or more prior tests and/or the subject's age, weight, height, ethnicity, medical history, a physical examination, an interview, a computerized test result, a medical image, a risk-related public health initiative or directive, etc. For example, a subject's risk profile may take into account that a target biomarker was identified as being present in a bodily fluid from a subject at a first time. Additionally, for example, a subject's risk profile may take into account that a target biomarker was identified as being present in a bodily fluid from a subject at a first time, and a second time, and so on.

In some embodiments of each or any of the above- or below-mentioned embodiments, the subject's risk profile is determined based upon the subject's membership in a class of subjects, wherein all subjects in the class are at risk of a common disease, disorder, or condition.

In some embodiments of each or any of the above- or below-mentioned embodiments, the target biomarker is a protein, a nucleic acid, a peptide, a polymer, an amino acid or a glycan.

In some embodiments of each or any of the above- or below-mentioned embodiments, the bodily fluid is blood, plasma, saliva, tears, urine, amniotic fluid, cerebrospinal fluid, pleural fluid, or peritoneal fluid.

In some embodiments of each or any of the above- or below-mentioned embodiments, the bodily fluid is blood.

In some embodiments of each or any of the above- or below-mentioned embodiments, the immuno-affinity inserts are magnetic.

In some embodiments of each or any of the above- or below-mentioned embodiments, the immuno-affinity inserts are fluorescent.

In some embodiments of each or any of the above- or below-mentioned embodiments, the immuno-affinity inserts comprise one of particles, tubes and filaments.

In some embodiments of each or any of the above- or below-mentioned embodiments, the bodily fluid is contacted with at more than one set of immuno-affinity inserts, and wherein each set is coated with a different affinity molecule.

In some embodiments of each or any of the above- or below-mentioned embodiments, the subject is predicted to have a cancer.

In some embodiments of each or any of the above- or below-mentioned embodiments, the cancer is non-melanoma skin cancer, lung cancer, breast cancer, prostate cancer, colorectal cancer, bladder cancer, melanoma, non-Hodgkin lymphoma, ovarian cancer, cervical cancer, or pancreatic cancer.

In some embodiments of each or any of the above- or below-mentioned embodiments, the subject is predicted to have Alzheimer's disease.

In some embodiments of each or any of the above- or below-mentioned embodiments, the target biomarker is tau protein.

In some embodiments of each or any of the above- or below-mentioned embodiments, the tau protein is hyperphosphorylated.

In some embodiments of each or any of the above- or below-mentioned embodiments, the affinity molecules are present in a soluble affinity matrix (SAM).

In some embodiments of each or any of the above- or below-mentioned embodiments, the SAM additionally comprises a preservative that protects a target molecule from degradation or biochemical modification.

In some embodiments of each or any of the above- or below-mentioned embodiments, a release of the set of immuno-affinity inserts from the SAM is triggered by wetness, pH or osmolality of the bodily fluid.

In some embodiments of each or any of the above- or below-mentioned embodiments, a first set of immuno-affinity inserts is present in a first layer of the SAM.

In some embodiments of each or any of the above- or below-mentioned embodiments, a second set of immuno-affinity inserts is present in a second layer of the SAM.

In some embodiments of each or any of the above- or below-mentioned embodiments, the SAM comprises a first layer that elutes a set of immuno-affinity inserts having affinity molecules that specifically bind one or more undesired biomarkers.

In some embodiments of each or any of the above- or below-mentioned embodiments, the SAM comprises a second layer that elutes the set of immuno-affinity inserts coated with affinity molecules specific for the target biomarker.

In some embodiments of each or any of the above- or below-mentioned embodiments, the first set of immuno-affinity inserts is released from the SAM at a first predetermined time and the second set of immuno-affinity inserts is released from the SAM at a second predetermined time.

In some embodiments of each or any of the above- or below-mentioned embodiments, the second predetermined time is later than the first predetermined time.

In some embodiments of each or any of the above- or below-mentioned embodiments, the affinity molecules are selected from the group consisting of antibodies, antibody fragments, enzymes, fragments of enzymes, enzyme substrates, fragments of enzyme substrates, nucleotides, oligonucleotides, polynucleotides, receptors, aptamers, nanobodies, fragments of receptors, ligands, fragments of enzymes, other proteins, amino acids, peptides, polypeptides, oligopeptides, saccharides, disaccharides, polysaccharides, glycoproteins, proteoglycans, and natural and synthetic polymers.

In some embodiments of each or any of the above- or below-mentioned embodiments, the SAM comprises immuno-affinity inserts comprising one of particles, tubes and filaments.

The present disclosure also provides a device for enriching a target biomarker from a bodily fluid of a subject comprising one or more sets of immuno-affinity inserts coated with affinity molecules specific for the target biomarker, wherein the affinity molecules are selected based on a risk profile for the subject.

In some embodiments of each or any of the above- or below-mentioned embodiments, the device further comprises i) a first component containing the one or more sets of immuno-affinity inserts and ii) a second component that retains immuno-affinity inserts bound to the target biomarker, wherein the first component is in fluid connection to the second component.

In some embodiments of each or any of the above- or below-mentioned embodiments, the first component is a cartridge.

In some embodiments of each or any of the above- or below-mentioned embodiments, the cartridge is selected by a computer controlled machine.

In some embodiments of each or any of the above- or below-mentioned embodiments, the cartridge is preloaded with the one or more sets of immuno-affinity inserts coated with affinity molecules specific for the target biomarker by a computer controlled machine.

In some embodiments of each or any of the above- or below-mentioned embodiments, the immuno-affinity inserts are magnetic.

In some embodiments of each or any of the above- or below-mentioned embodiments, the second component contains a magnetic element to capture the immuno-affinity inserts.

In some embodiments of each or any of the above- or below-mentioned embodiments, the magnetic element is reusable.

In some embodiments of each or any of the above- or below-mentioned embodiments, the magnetic element is located on the outside surface of the second component.

In some embodiments of each or any of the above- or below-mentioned embodiments, the affinity molecules are selected from the group consisting of antibodies, antibody fragments, enzymes, fragments of enzymes, enzyme substrates, fragments of enzyme substrates, nucleotides, oligonucleotides, polynucleotides, receptors, aptamers, nanobodies, fragments of receptors, ligands, fragments of enzymes, other proteins, amino acids, peptides, polypeptides, oligopeptides, saccharides, disaccharides, polysaccharides, glycoproteins, proteoglycans, and natural and synthetic polymers.

In some embodiments of each or any of the above- or below-mentioned embodiments, there are at least two sets of paramagnetic immuno-affinity inserts, wherein each set of paramagnetic immuno-affinity inserts is coated with a different affinity molecule, and wherein each set of paramagnetic immuno-affinity inserts is marked with a different fluorescent dye or combination of fluorescent dyes.

In some embodiments of each or any of the above- or below-mentioned embodiments, the paramagnetic immuno-affinity inserts are continually released from the first component as the bodily fluid flows through the first component.

In some embodiments of each or any of the above- or below-mentioned embodiments, the target biomarker is predicted to be present in the bodily fluid based on the subject's risk profile.

In some embodiments of each or any of the above- or below-mentioned embodiments, the subject's risk profile is based on age, weight, height, ethnicity, medical history, a physical examination, a patient interview, a computerized test result, a medical image, a risk-related public health initiative or directive, or any combination thereof. In a preferred embodiment, the subject's risk profile is based on whether one or more target biomarkers were identified as being present or absence in a bodily fluid from the subject in one or more prior tests and/or the subject's age, weight, height, ethnicity, medical history, a physical examination, an interview, a computerized test result, a medical image, a risk-related public health initiative or directive, etc. For example, a subject's risk profile may take into account that a target biomarker was identified as being present in a bodily fluid from a subject at a first time. Additionally, for example, a subject's risk profile may take into account that a target biomarker was identified as being present in a bodily fluid from a subject at a first time, and a second time, and so on.

In some embodiments of each or any of the above- or below-mentioned embodiments, the subject's risk profile is determined based upon the subject's membership in a class of subjects, wherein all subjects in the class are at risk of a common disease, disorder, or condition.

In some embodiments of each or any of the above- or below-mentioned embodiments, the device further comprises a computer controlled machine that selects immuno-affinity inserts.

In some embodiments of each or any of the above- or below-mentioned embodiments, the immuno-affinity inserts are filaments.

In some embodiments of each or any of the above- or below-mentioned embodiments, the filaments are coiled, compound-coiled, straight or matted.

In some embodiments of each or any of the above- or below-mentioned embodiments, the filaments are a fiber, a micro-tubule or a nanotube.

In some embodiments of each or any of the above- or below-mentioned embodiments, the device further comprises a filament structure having a plurality of filaments attached together in a horsetail configuration.

In some embodiments of each or any of the above- or below-mentioned embodiments, the device further comprises a filament structure having a plurality of filaments bound or wound to a central support structure.

The present disclosure also provides computer-implemented methods, comprising, at a server system including one or more processors and memory storing one or more programs for execution by the one or more processors: predicting, based on a predictive risk model, that a target biomarker is present in bodily fluid of a first subject; receiving first assay data including identification of presence or lack of presence of the target biomarker; and updating the predictive risk model, based on the first assay data.

In some embodiments of each or any of the above- or below-mentioned embodiments, the predictive risk model is based on stored health data of the first subject including one or more of: medical history data, environmental data, behavioral data, genomic data, vitals data, biometrics data, and/or a medical image of the first subject.

In some embodiments of each or any of the above- or below-mentioned embodiments, the predictive risk model is based on stored health data of a plurality of subjects including one or more of: medical history data, environmental data, behavioral data, genomic data, vitals data, biometrics data, and/or medical images of the plurality of subjects.

In some embodiments of each or any of the above- or below-mentioned embodiments, the predictive risk model is further based on general public health data, including one or more of: a health directive or initiative, medical research, and/or health data based on causal trends or events.

In some embodiments of each or any of the above- or below-mentioned embodiments, updating the predictive risk model includes: associating the first assay data with the stored health data of the first subject; and training the predictive risk model using the stored health data of the first subject as input data and the first assay data as an output label for the input data.

In some embodiments of each or any of the above- or below-mentioned embodiments, training the predictive risk model includes using supervised training, unsupervised training, and/or adversarial training to associate subsequently stored health data with the first assay data if the subsequently stored health data is similar to the previously stored health data.

In some embodiments of each or any of the above- or below-mentioned embodiments, associating the subsequently stored health data with the first assay data includes storing an indication of predicted presence or lack of presence of the target biomarker in relation to the subsequently stored health data.

In some embodiments of each or any of the above- or below-mentioned embodiments, the methods further comprise predicting, based on the updated predictive risk model, that the target biomarker is present in bodily fluid of a second subject.

In some embodiments of each or any of the above- or below-mentioned embodiments, the methods further comprise predicting, based on the updated predictive risk model, that the target biomarker is present in a subsequently obtained sample of bodily fluid of the first subject.

In some embodiments of each or any of the above- or below-mentioned embodiments, the methods further comprise predicting, based on the updated predictive risk model, that the target biomarker is no longer present in bodily fluid of the first subject.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 is a graphic illustration of a risk profile.

FIG. 2 is a partial cross-sectional view of a soluble affinity matrix (SAM).

FIG. 3 is a perspective cross-sectional view of a fluid flow tube containing affinity molecule bearing beads.

FIG. 4A is a cross-sectional view of a fluid flow tube.

FIG. 4B is a cross-sectional view of the fluid flow tube of FIG. 4A containing beads having a toroidal structure.

FIG. 4C is a cross-sectional view of the fluid flow tube of FIG. 4A containing beads having an ovoid structure.

FIG. 5 is a schematic view of a bodily fluid collection assembly.

FIG. 6 is a perspective view of a bodily fluid vessel containing a filament structure.

FIGS. 7A-7C are system diagrams of a computing system in accordance with some embodiments.

FIG. 8 is a schematic diagram of a method of predictive risk calculation in accordance with some embodiments.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the claims. Various aspects of the subject technology will now be disclosed according to particular but non-limiting examples. Various embodiments described in the present disclosure may be carried out in different ways and variations, and in accordance with a desired application or implementation.

In the following detailed description, numerous specific details are set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

The separation of low abundance proteins in the blood and the determination of their identity is often confounded by the presence of other highly abundant plasma proteins. The present disclosure advantageously provides methods for the enrichment of a target biomarker from a biological fluid. Such methods may selectively enrich a target biomarker that may be present or is predicted to be present in the biological fluid. The enrichment of the target biomarker may result from the sequestering of the target biomarker present in the biological fluid and/or the depletion of unwanted biomarkers present in the biological fluid. The methods disclosed herein uniquely provide for the early detection and diagnosis of diseases and/or disorders (e.g., diseases and/or disorders characterized by the presence of a biomarker in a bodily fluid including, for example, a low abundance biomarker) such as cancer as well as provide for monitoring the progress of the disease or disorder and for monitoring the progress of a therapy (e.g., anticancer therapy) used to treat the disease or disorder.

In an embodiment, two or more devices each comprising immuno-affinity inserts as disclosed herein may be used to enrich a target biomarker present in a biological fluid. After contacting the biological fluid the target biomarker may be released from the immuno-affinity inserts and pooled for analysis (e.g., identification of the presence or absence of the target biomarker and/or its abundance in the biological fluid).

In the methods disclosed herein, a risk profile of the subject is used to establish the identity of a target biomarker that is likely to be present in the bodily fluid so that the target biomarker can be selectively enriched from the bodily fluid. In this manner, a personalized panel of biomarkers is enriched from the subject thereby reducing noise caused by unwanted biomarkers or irrelevant biomarkers. The methods disclosed herein advantageously reduce the costs associated with the identification of a target biomarker by eliminating irrelevant or undesired biomarkers (e.g., those biomarkers not associated with diseases or disorders that the subject may be predisposed to developing).

The risk profile may indicate medical diseases, disorders, or conditions that the subject is likely to develop including those diseases, disorders, or conditions that afflict subjects of a similar disposition (e.g., subjects of a similar gender, age, weight, height, ethnicity, medical history, a physical examination, a patient interview, a computerized test result, a medical image, a risk-related public health initiative or directive, or any combination thereof). For example, if the subject is an elderly individual that individual may be at risk of a neurodegenerative disease such as Alzheimer's disease. Consequently, the tau protein (or a hyperphosphorylated form of tau protein) may be selected as a target biomarker.

In another example, the subject may be at risk of cancer, such as oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, glioma, parathyroid cancer, penile cancer, vaginal cancer, thyroid cancer, pancreatic cancer, esophageal cancer, Hodgkin's lymphoma, leukemia-related disorders, mycosis fungoides, myelodysplastic syndrome, non-small cell lung cancer, pancreatic cancer, breast cancer, ovarian cancer, colorectal cancer, head and neck cancer, a carcinoma, a tumor, a neoplasm, a lymphoma, a melanoma, a glioma, a sarcoma, or a blastoma.

The listing of a subject's cancer risk may be even more granular. For example, a carcinoma may include any of adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiated carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

Similarly, a tumor may include any of astrocytic tumors, malignant mesothelial tumors, ovarian germ cell tumors, supratentorial primitive neuroectodermal tumors, Wilms tumors, pituitary tumors, extragonadal germ cell tumors, gastrinoma, germ cell tumors, gestational trophoblastic tumors, brain tumors, pineal and supratentorial primitive neuroectodermal tumors, pituitary tumors, somatostatin-secreting tumors, endodermal sinus tumors, carcinoids, central cerebral astrocytoma, glucagonoma, hepatic adenoma, insulinoma, medulloepithelioma, plasmacytoma, vipoma, and pheochromocytoma.

Still further, a neoplasm may include any of intraepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma. In a further embodiment the lymphoma may be selected from the group consisting of: nervous system lymphoma, AIDS-related lymphoma, cutaneous T-cell lymphoma, non-Hodgkin's lymphoma, lymphoma, and Waldenstrom's macroglobulinemia. In another embodiment the melanoma may be selected from the group consisting of: acral lentiginous melanoma, superficial spreading melanoma, uveal melanoma, lentigo maligna melanomas, melanoma, intraocular melanoma, adenocarcinoma nodular melanoma, and hemangioma. In yet another embodiment the sarcoma may be selected from the group consisting of: adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma. In one embodiment the glioma may be selected from the group consisting of: glioma, brain stem glioma, and hypothalamic and visual pathway glioma. In another embodiment the blastoma may be selected from the group consisting of: pulmonary blastoma, pleuropulmonary blastoma, retinoblastoma, neuroblastoma, medulloblastoma, glioblastoma, and hemangiblastomas.

The risk profile may be determined based upon the subject's age, weight, height, ethnicity, medical history, a physical examination, a patient interview, a computerized test result, a medical image, a risk-related public health initiative or directive, or any combination thereof. Accordingly, the risk profile may be personalized for a specific group of subjects (e.g., male, non-smoker, over 40 years old) or even a specific individual subject (e.g., John Doe).

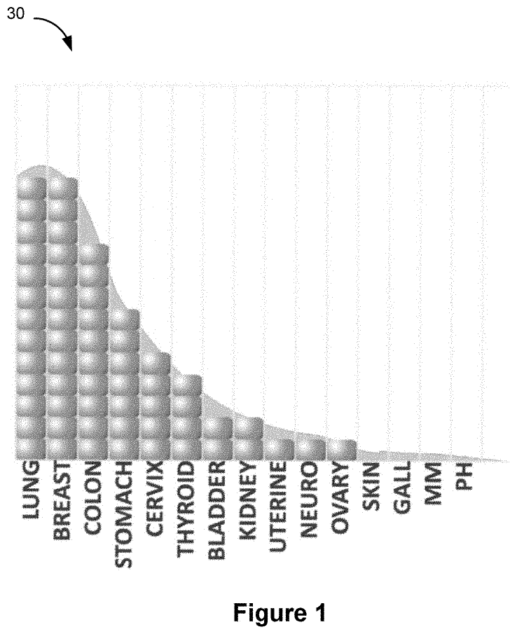

FIG. 1 shows a graphical risk profile 30 for a subject. In this example, the subject is a female and the risk profile 30 indicates that the subject is at varying levels of risks of cancers such as lung, breast, colon, stomach, cervical, thyroid, bladder, kidney, uterine, neuroendocrine, or ovarian cancer. In this manner, biomarkers for irrelevant diseases, disorders, or conditions such as prostate cancer may be excluded from the risk profile 30. Further, the varying levels of risk shown in the risk profile 30 provide for the ability to zero in on target biomarkers for the most relevant risk factors. For example, a biomarker assay for the risk profile 30 in FIG. 1 may only include target biomarkers for the top three risk categories of lung, breast and colon cancer in order to reduce costs and time associated with testing for all of the disorders shown in the risk profile 30.

A risk profile may indicate risk factors in percentage terms. For example, a subject's risk profile may indicate that the likelihood of developing a particular disease, disorder, or condition is 50% or greater, 55% or greater, 60% or greater, 65% or greater, 70% or greater, 75% or greater, 80% or greater, 85% or greater, 90% or greater, or 95% or greater.

The risk profile may be used for accurately and efficiently enriching a target biomarker from a bodily fluid (e.g., blood, plasma, saliva, tears, urine, amniotic fluid, cerebrospinal fluid, pleural fluid, or peritoneal fluid) of a subject. For example, the risk profile may be used in predicting that a target biomarker is or may be present in the bodily fluid of the subject. Based on the prediction, the subject's bodily fluid may be contacted with a set of immuno-affinity inserts coated with affinity molecules specific for the target biomarker under conditions for the affinity molecules to bind the target biomarker, optionally followed by separating the affinity molecules bound to the target biomarker from the bodily fluid.

In an embodiment, the risk profile may be used to identify an appropriate number of affinity molecules required to bind an amount (e.g., 20%, 25%, 30%, 35%, 40%, 45%, 50%, 55%, 60%, 65%, 70%, 75%, 80%, 85%. 90%, 95%, 99%, or 100%) of a target biomarker in a biological sample. The number of affinity molecules required to bind an amount of a target biomarker may be used to select a number (e.g., an optimal number) of immuno-affinity inserts for use in the disclosed methods. For example, where a subject is predicted to have a high likelihood of having or developing lung cancer a number of immune-affinity inserts coated with affinity molecules specific for a target biomarker associated with lung cancer may be selected so that the target biomarker (e.g., present in a given amount of a bodily fluid) binds to 50% of the affinity molecules on the immuno-affinity inserts specific for the target biomarker.

For example, based on the risk profile 30 of FIG. 1, the process for enriching target biomarkers from a bodily fluid of the female subject may begin by determining that only biomarkers for lung and breast cancer are to be targeted based on those being the highest risk shown on the risk profile 30. The blood of the female subject is obtained and flowed through a first component containing two sets of particles coated with affinity molecules specific for the target biomarkers, one set targeted to lung cancer biomarkers and another set targeted to breast cancer biomarkers. The blood is then flowed through a second component that retains the particles bound to the target biomarkers. In this example, the second component may be a single component that retains both the particles bound to the lung cancer biomarkers and the breast cancer biomarkers, or there may be two second components, one for each target biomarker. The retained biomarkers may then be measured and/or tested to provide information on whether the female subject has either lung or breast cancer, and potentially the severity of the disease if present.

In some aspects the particles are inert. In some aspects the particles are magnetic such that the magnetic particles can be collected by using a magnetic field. In some aspects the particles are fluorescent such that the fluorescent particles can be identified, measured and/or counted visually.

Thus, the devices and methods in the present disclosure provide for diagnosing a subject with a disease, disorder, or condition based on the target biomarker for the disease, disorder, or condition being identified in the subject's bodily fluid. Put another way, the target biomarker may be enriched from the subject's bodily fluid by the disclosed devices and methods, wherein the enriched biomarker may be used to diagnose the presence or absence of a disease, disorder, or condition. In addition, the enriched biomarker may also be used to monitor the advancement of a disease, disorder, or condition, as well as to monitor the effectiveness of a treatment regimen.

For example, “treating” or “treatment” of a disease, disorder, or condition may include at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms. The treating or treatment of a disease or disorder may include treating or the treatment of cancer.

The term “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

An “effective amount,” as used herein, refers to the amount of an active composition that is required to confer a therapeutic effect on the subject. A “therapeutically effective amount,” as used herein, refers to a sufficient amount of an agent or a compound being administered which will relieve to some extent one or more of the symptoms of the disease, disorder, or condition being treated. In some embodiments, the result is a reduction and/or alleviation of the signs, symptoms, or causes of a disease, or any other desired alteration of a biological system. For example, in some embodiments, an “effective amount” for therapeutic uses is the amount of the composition including a compound as disclosed herein required to provide a clinically significant decrease in disease symptoms without undue adverse side effects. In some embodiments, an appropriate “effective amount” in any individual case is determined using techniques, such as a dose escalation study. The term “therapeutically effective amount” includes, for example, a prophylactically effective amount. In other embodiments, an “effective amount” of a compound disclosed herein, such as a compound of Formula (A) or Formula (I), is an amount effective to achieve a desired pharmacologic effect or therapeutic improvement without undue adverse side effects. In other embodiments, it is understood that “an effective amount” or “a therapeutically effective amount” varies from subject to subject, due to variation in metabolism, age, weight, general condition of the subject, the condition being treated, the severity of the condition being treated, and the judgment of the prescribing physician.

As discussed above, target biomarkers may be bound by affinity molecules that are exposed to the bodily fluid. The affinity molecules may be coated onto any suitable surface, structure or substrate to create an immuno-affinity insert. For example, affinity molecules may be disposed on or bonded to (e.g., covalently bonded) beads (e.g., microspheres), fibers, microtubes, nanotubes, the inner lining of a fluid flow tube, and the like. The underlying structure may host one single type of affinity molecule for binding one specific biomarker, or several types of affinity molecules, each one for binding a different biomarker.

In some aspects, the bodily fluid is contacted with two or more immuno-affinity inserts. In a further aspect, at least two of the two or more immuno-affinity inserts are coated with affinity molecules specific for different target biomarkers (e.g., one immuno-affinity insert is coated with affinity molecules specific for target biomarker A and the other immuno-affinity insert is coated with affinity molecules specific for target biomarker B). In an aspect, the two or more immuno-affinity inserts coated with different affinity molecules are compatible with one another (e.g., bind to target biomarkers under similar conditions including temperature, pH, etc. using similar reagents such as buffers etc.

The bodily fluid may be contacted with a device comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more immuno-affinity inserts. In a further embodiment, the bodily fluid is contacted with 50, 60, 70, 80, 90, 100, 110, 120, 130, 140, 150, 160, 170, 180, 190, 200, or more immuno-affinity inserts. All of the immuno-affinity inserts may be the same (e.g., be coated with the same affinity molecules). Alternatively, the bodily fluid may be contacted with a combination of immuno-affinity inserts where some or all of the immuno-affinity inserts may be different (e.g., be coated with different affinity molecules). In other embodiments, an affinity insert may be coated with two or more different affinity molecules each binding to a different target biomarker (e.g., the affinity insert is coated with a plurality of affinity molecules that bind target biomarker A and a plurality of affinity molecules that bind target biomarker B).

In an aspect, the number and combination of immuno-affinity inserts may be based on the affinity and/or avidity of the affinity molecules. In another aspect, the number and/or combination of immuno-affinity inserts may be based upon an incidence of the one or more target biomarkers per unit volume of the bodily fluid.

The immuno-affinity inserts may be a unit quantity of liquid, semi liquid colloidal suspension or gel or any combination thereof.

The number and combination of immuno-affinity inserts may be based upon a requirement for the bodily fluid to remain viable for other tests such as blood gas, glucose, blood chemistry, western blot, etc. Additionally or alternatively, the number and/or combination of immuno-affinity inserts is based upon the detectability characteristics of the one or more target biomarkers including, for example, the signal to noise ratio for each of the one or more target biomarkers. Additionally or alternatively, the number and combination of immuno-affinity inserts are based upon mitigating non-specific binding of a target biomolecule to an affinity molecule specific for a different target biomolecule.

Target biomarkers bound to an immuno-affinity insert may be released from the immuno-affinity insert and subjected to further analysis (e.g., identification of the presence or absence of the target biomarkers and/or its abundance in the biological fluid). The presence of absence and/or the abundance of the target biomarker may indicate the presence of a disease or disorder or a predisposition for developing the disease or disorder.

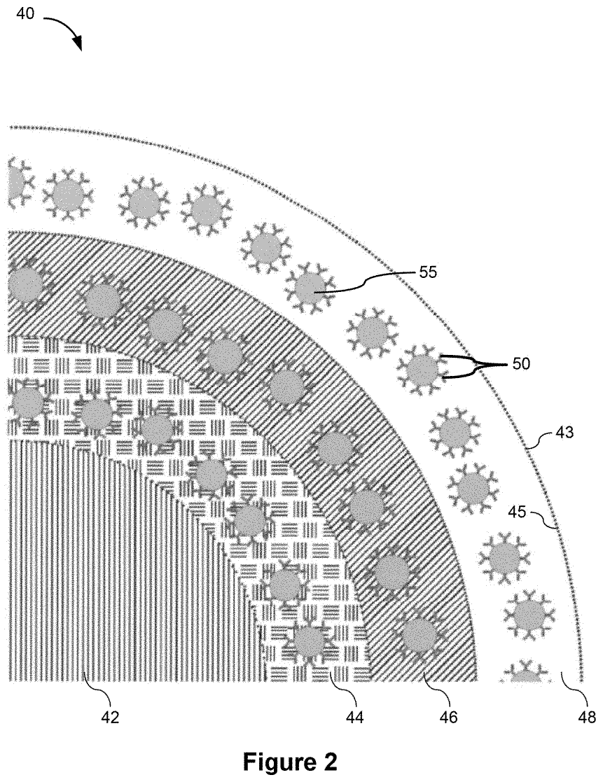

For example, FIG. 2 shows a partial cross-sectional view of a soluble affinity matrix (SAM) 40 in the form of a multilayered bead, fiber, tube, etc. and containing one or more types of affinity molecules 50. The affinity molecules 50 may be connected, bonded or coated to particles 55. An inner core 42 may be a material that contains no affinity molecules. For example, the inner core 42 of a bead or a fiber may be a solid material, while the inner core 42 of a tube (e.g., nanotube, fluid collecting/transfer tube) may be a hollow space that may or may not be open to fluid flow. The SAM 40 shown in FIG. 2 has successive material layers 44, 46, 48 that each contain multiple particles 55 each having multiple affinity molecules 50. Here, each material layer 44, 46, 48 may have the same type of affinity molecule 50 (e.g., structured to bind lung cancer biomarkers) or each material layer 44, 46, 48 may have a different type of affinity molecule 50 (e.g., layer 44 structured to bind lung cancer biomarkers, layer 46 structured to bind stomach cancer biomarkers, layer 48 structured to bind pancreas cancer biomarkers). In another example, each layer 44, 46, 48 may contain a mixture of different affinity molecules 50. In another example, the inner core 42 of a tube (e.g., nanotube, fluid collecting/transfer tube) may be connected, bonded, or coated with affinity molecules 50.

In the example of FIG. 2, a set of particles 55 having affinity molecules 50 in each layer 44, 46, 48 are released from the SAM 40 based on solubility (e.g., dissolving) of each successive layer 44, 46, 48. The solubility of layers 44, 46, 48 may be based on one or more properties of the bodily fluid, such as wetness, pH or osmolality. For example, if SAM 40 is a bead or fiber immersed in a tube of blood, then layer 48 would initially be the only layer in contact with the blood and would dissolve to release the particles 55 having affinity molecules 50 contained in layer 48. The blood then comes into contact with layer 46, subsequently releasing the particles 55 having affinity molecules 50 contained in layer 46, after which the blood contacts layer 44, thus releasing the particles 55 having affinity molecules 50 contained in layer 44. Thus, the staggered timing of particle 55 release allows for releasing different sets of affinity molecules 50 at different times, releasing mixed sets of affinity molecules 50 at different times, or for releasing multiple waves of similar affinity molecules 50 (e.g., time release). Any particular set of particles 55 may be released at a predetermined time, allowing for release of different sets of particles 55 at different times.

Continuing with the SAM 40 structure shown in FIG. 2, if the inner core 42 is hollow, then the bodily fluid may flow through the hollow inner core 42 and the layers 44, 46, 48 would dissolve in the opposite order as described above. Here, the SAM 40 may have an outer surface 43 that is a tube wall, such as a tube for collecting or flowing blood. Thus, layer 48 is deposited on an inner surface 45 of outer surface 43, layer 46 deposited on layer 48 and layer 44 deposited on layer 46.

In yet another example, a hollow inner core 42 and an outer surface 43 of the SAM 40 may both be exposed to the bodily fluid simultaneously. Here, the outer surface 43 is the outer surface of dissolvable layer 48. Thus, this structure can be seen as a dissolvable straw or tube that allows for dissolving of layers 44, 46, 48 from both inner and outer directions, wherein the straw or tube is gone once the layers 44, 46, 48 are completely dissolved.

A SAM 40 may have any number of layers containing affinity molecules 50. For example, a SAM 40 may have a single layer containing one or multiple types of affinity molecules 50. A SAM 40 may also two or four or more layers as well.

In any of the SAM 40 structures described above, the dissolving of layers 44, 46, 48 may result in releasing the particles 55 having affinity molecules 50 into the bodily fluid to bind with target biomarkers in the bodily fluid. The resulting particles 55 with bound affinity molecules 50 may then be collected or filtered to be tested and/or to enrich the remaining bodily fluid by removal of undesired molecules. For example, any of the layers 44, 46, 48 may elute a set of particles 55 having affinity molecules 50 that specifically bind undesired biomarkers, while any other layer 44, 46, 48 may elute a set of particles 55 having affinity molecules 50 that specifically bind target biomarkers.

A SAM 40 may include affinity molecules 50 contained directly within the soluble layer 44, 46, 48 and that are not connected to a particle. Here, each affinity molecule 50 may be independently released into the bodily fluid from the dissolving layer 44, 46, 48 and bind to a target biomarker. The resulting independent bound affinity molecules 50 may then be collected or filtered to be tested and/or to enrich the remaining bodily fluid by removal of undesired molecules. Any method, step, process, etc. discussed above or below regarding particles 55 may be applied to independent affinity molecules 50.

A SAM 40 may also include a preservative that protects a target biomarker from degradation of biochemical modification. The preservative may protect one particular type of biomarker or a group or class of biomarkers. For example, the SAM 40 shown in FIG. 2 may include multiple preservatives, each one provided to protect target biomarkers bound by the affinity molecules 50 of a specific layer 44, 46, 48.

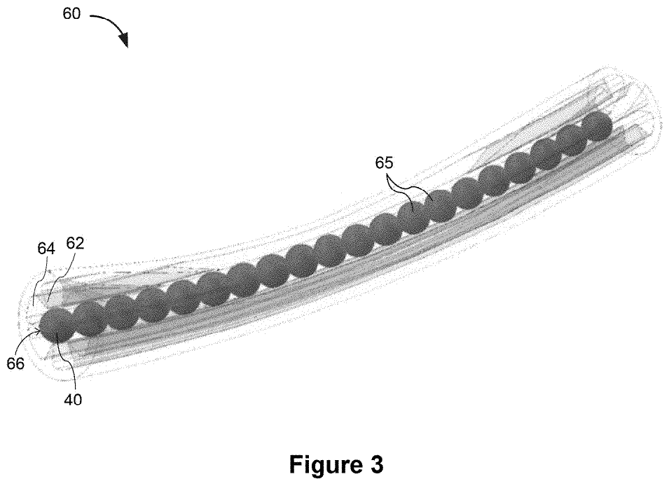

FIG. 3 shows an example of a fluid flow tube 60 (e.g., blood collection tubing) containing multiple beads (e.g., microspheres) 65 arranged in single file through the middle of the fluid flow tube 60. Multiple ridges 62 are disposed on the inner surface 64 of the tube 60. Here, bodily fluid can flow through the areas between the ridges 62 while contacting the outer surface of the beads 65. In some aspects, the beads 65 may be non-soluble beads that have affinity molecules coated or bonded to the bead surface. In this case, as the bodily fluid flows past the beads 65, target biomarkers in the bodily fluid bind to the affinity molecules on the beads 65, after which the beads 65 may be removed from the fluid flow tube 60 and further processed as desired.

In some aspects, the beads 65 may be formed of soluble materials, such as the above-described SAM 40 structure. Here, as the bodily fluid flows past the beads 65, affinity molecules on the beads 65 are released into the bodily fluid and target biomarkers in the bodily fluid bind to the affinity molecules, after which the bodily fluid may be collected and the affinity molecules within the collected bodily fluid may be processed as desired. Further, as the one or more outer layers 44, 46, 48 of the soluble beads 65 dissolve, fluid flow volume in the central portion 66 of the tube 60 may increase. The soluble beads 65 may be configured to dissolve completely within the bodily fluid or to have an inner core 42 that is not soluble. The non-soluble cores 42 may be collected or filtered out of the bodily fluid, or may remain within the processed bodily fluid.

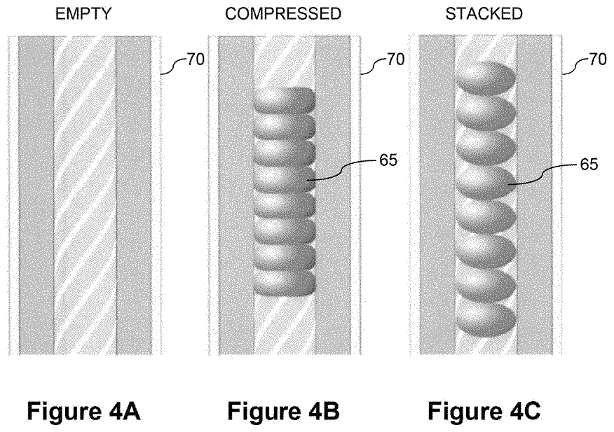

FIG. 4A shows an example of an empty fluid flow tube 70, such as clear plastic or silicone tubing used in disposable blood collection sets. FIG. 4B shows an example of the fluid flow tube 70 containing a set of beads 65 (e.g., SAMs 40) configured as compressed or toroidal shaped structures. Similarly, FIG. 4C shows an example of the fluid flow tube 70 containing a set of beads 65 configured as oval or egg-shaped structures. It is contemplated that a bead 65 structure or SAM 40 structure may be any desirable shape. The size and or shape of the bead 65 or SAM 40 may be determined by any combination of relevant parameters, such as the type of affinity molecules 50 to be used, the desired bodily fluid flow rate, the dissolving time of a soluble layer and a binding time for a target biomarker, for example.

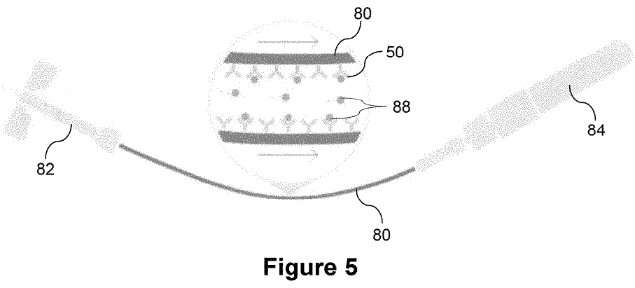

Affinity molecules 50 may also be coated or attached directly to a fluid flow tube 80 as shown in FIG. 5. Here, the fluid flow tube 80 is a blood collection tube that connects a needle assembly 82 to a blood collection tube 84. As blood flows through the tube 80, the blood comes into contact with the affinity molecules 50 lining an inner surface 86 of the tube 80. The affinity molecules 50 each bind a particular type of biomarker 88, thus removing that biomarker 88 from the blood flow. Thus, the removal of the bound biomarkers 88 from the blood flow may be used to enrich the blood collected in the collection tube 84 by removing undesirable blood components, for example. As another example, once the final blood product is collected in the collection tube 84 and the collection tube 84 is disconnected from the flow tube 80, the flow tube 80 can be flushed with a solution that unbinds the biomarkers 88, thus allowing the released biomarkers 88 to be collected for testing or further processing.

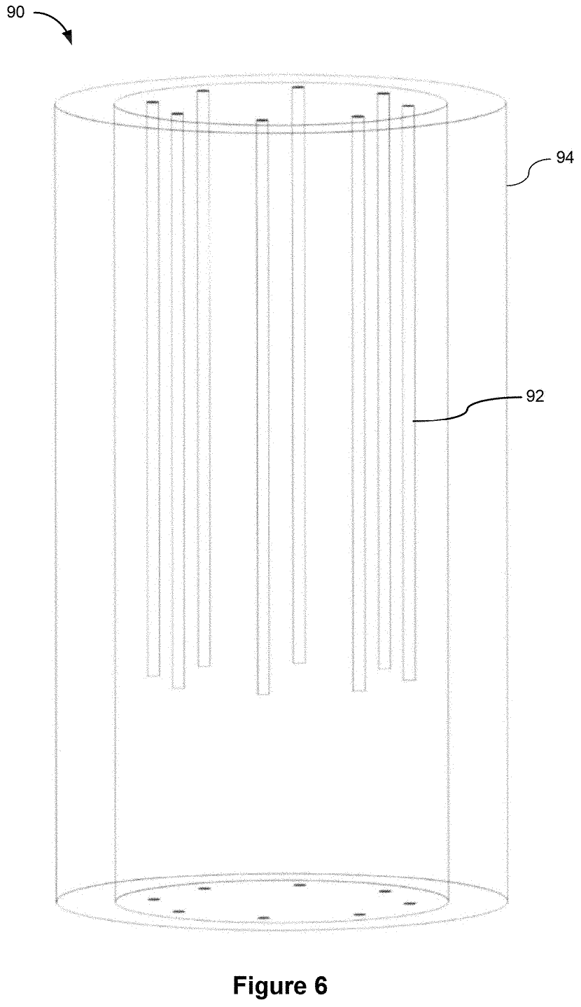

FIG. 6 shows an example of a filament structure 90 having multiple filaments 92. The filaments 92 may be fibers, micro-tubules, nanotubes or any other desirable structure for hosting affinity molecules. As shown in FIG. 6, the filaments 92 are straight tubes, though any other desired structure may be used (e.g., coiled, compound-coiled, matted). The filaments 92 may be bundled together at one end in a horsetail configuration, connected in a branch configuration, bound and/or wound to a central structure comparable to a pipe-cleaner, for example, to form the filament structure 90. In use, the filament structure 90 may be inserted into a bodily fluid containing vessel 94 (e.g., a blood collection tube including, for example, a capillary blood collection tube) and subsequently removed from the vessel 94 after a period of time. In some embodiments, the immersed filaments 92 may be coated with affinity molecules that bind particular biomarkers, which are then removed from the bodily fluid when the filament structure 90 is removed from the vessel 94. In some embodiments, the filaments 92 may be configured as SAM 40 structures that partially or fully dissolve when inserted into the bodily fluid in the vessel 94, thereby releasing one or more types of affinity molecules into the bodily fluid contained within the vessel 94.

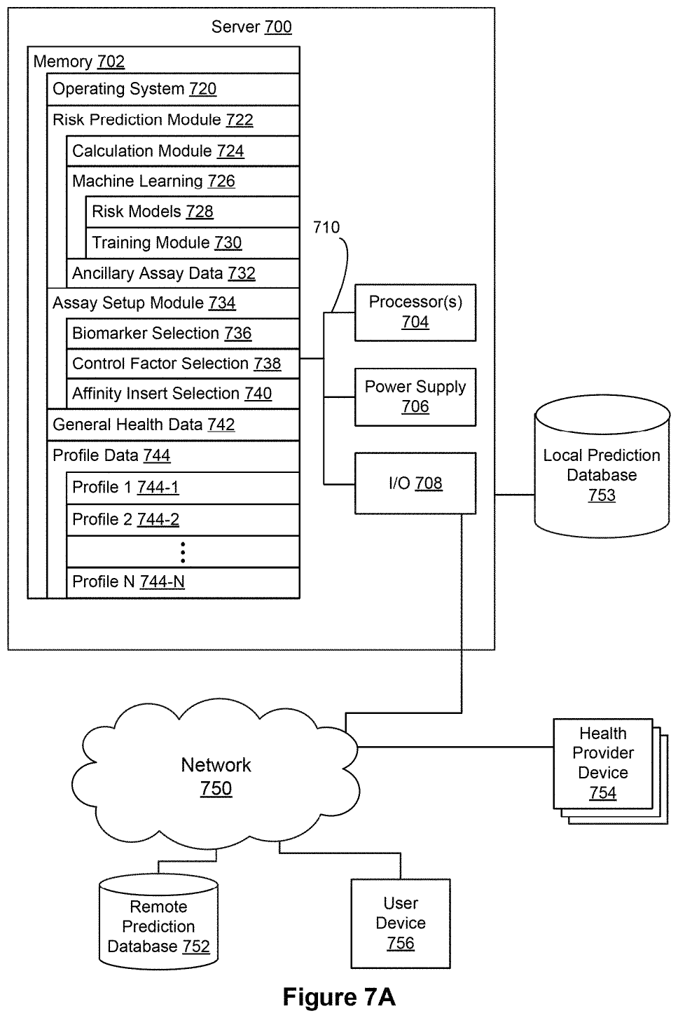

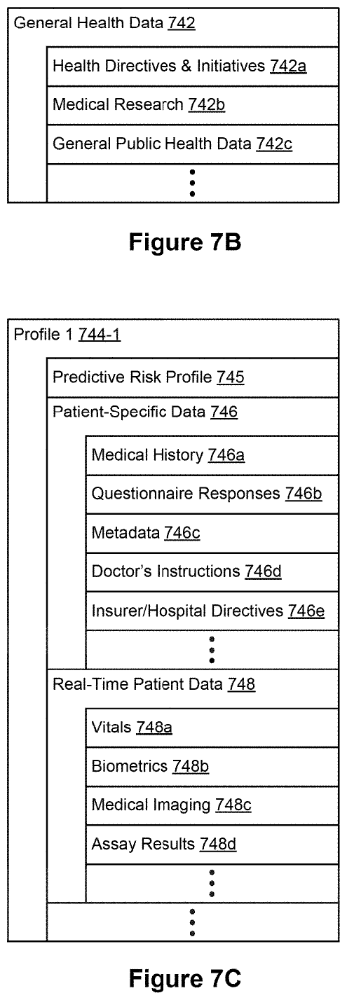

FIGS. 7A-7C are system diagrams of a server 700 (alternatively referred to herein as a “diagnosis system,” “computing system,” or a “system”), in accordance with some embodiments. The server 700 typically includes a memory 702, one or more processors 704, a power supply 706, an input/output (I/O) subsystem 708, and a communication bus 710 for interconnecting these components.

The processor(s) 704 execute modules, programs, and/or instructions stored in the memory 702 and thereby perform processing operations.

In some embodiments, the memory 702 stores one or more programs (e.g., sets of instructions) and/or data structures, collectively referred to as “modules” herein. In some embodiments, the memory 702, or the non-transitory computer readable storage medium of the memory 702 stores the following programs, modules, and data structures, or a subset or superset thereof:
an operating system 120;
a risk prediction module 722 which determines personalized risk predictions for one or more patients based on predictive risk models derived for respective patients, the risk prediction module including:
   a calculation module 724 which calculates probabilities of presence of particular biomarkers in bodily fluid of particular patients;
   a machine learning module 726 for evaluating predictions calculated by the risk prediction module by comparing the predictions with corresponding test results and updating one or more risk models 728 and, in some embodiments, updating the risk models using a training module 730 (e.g., using machine learning); and
   ancillary assay data 732, including the test results used by the machine learning module 726 for evaluating and updating the risk models 728;
an assay setup module 734 for determining assay testing parameters, such as target biomarkers control factors, and affinity inserts, the assay setup module 734 including:
   a biomarker selection module 736 for determining one or more target biomarkers associated with a particular risk profile, as described above with regard to the example risk profile 30 (e.g., determining to target the biomarkers associated with the three highest risk diseases as determined by the risk prediction module 722, or any number of the highest risk diseases);
   a control factor selection module 738 for determining one or more control factors or confounding factors to be immuno-captured, measured, not measured, buffered, factored out, or otherwise designated and/or validated in accordance with a particular risk profile and/or prediction; and
   an affinity insert selection module 740 for determining which types of immuno-affinity inserts, how many of each type of immuno-affinity inserts, and/or what order the immuno-affinity inserts are to be disposed or used in a particular assay in accordance with a particular risk profile and/or prediction.

The above identified modules (e.g., data structures and/or programs including sets of instructions) need not be implemented as separate software programs, procedures, or modules, and thus various subsets of these modules may be combined or otherwise re-arranged in various embodiments. In some embodiments, the memory 702 stores a subset of the modules identified above. In some embodiments, a remote prediction database 752 and/or a local prediction database 753 store a portion or all of one or more modules identified above (such as the calculation module 724, the risk models 728, and/or the training module 730). Furthermore, the memory 702 may store additional modules not described above. In some embodiments, the modules stored in the memory 702, or a non-transitory computer readable storage medium of the memory 702, provide instructions for implementing respective operations in the methods described below. In some embodiments, some or all of these modules may be implemented with specialized hardware circuits that subsume part or all of the module functionality. One or more of the above identified elements may be executed by one or more of the processor(s) 704. In some embodiments, one or more of the modules described with regard to the memory 702 is implemented in the memory of a health provider computing device 754 (e.g., a computing device of a medical provider in the context of a doctor/patient, the patient being the subject of the risk prediction(s) and assay testing), and executed by processor(s) of the provider device 754. In some embodiments, one or more of the modules described with regard to the memory 702 is implemented in the memory of a user device 756 (e.g., a computing device of the patient subject to the risk prediction(s) and assay testing) and executed by processor(s) of the user device 756. For example, machine learning module 726 is distributed across server 700, provider device(s) 754, and/or user device 756.

In some embodiments, for each prediction process, machine learning module 726 stores data for training risk model(s) 728, such as:
general health data 742 corresponding to a plurality (e.g., a population) of individuals, including:
   health directives and initiatives 742a (e.g., from the Centers for Disease Control),
   medical research 742b (e.g., pertaining to risk correlations), and/or
   general public health data 742c (e.g., causal trends or events);
profile data 744 corresponding to one or more individual patients (e.g., personalized, patient-specific risk data), and/or corresponding to one or more types of patients (e.g., generic risk data), including:
   a predictive risk profile 745 (e.g., profile 30 of FIG. 1), including probabilities that certain diseases or conditions are present in the patient or would be present in the type of patient indicated by the particular profile;
   patient specific data 746 (or patient type-specific data), including data associated with a medical history, questionnaire responses, environmental, behavior, genomic, and/or additional metadata, doctor's instructions, and/or insurer or hospital directives; and/or
   real-time patient data 748, including vitals, biometrics, medical imaging, and/or assay results.

In some embodiments, generating a risk model 728 includes generating a regression algorithm for prediction of continuous variables (e.g., patent-specific data 746 and real-time patent data 748 gathered during subsequent doctor visits).

In some embodiments, the I/O subsystem 708 communicatively couples the server 700 to one or more devices, such as a local prediction database 753, a remote prediction database 752, a health provider computing device 754, and/or a user device 756 via a communications network 750 and/or via a wired and/or wireless connection. In some embodiments, the communications network 750 is the Internet. In some embodiments, the server 700 is a computing system local to a health facility at which the health provider and/or the user (also referred to herein as a patient or subject) is located. In some embodiments, the server 700 is remotely located with regard to the location of the health provider and/or the user. In some embodiments, one or more, or all, of the modules, components, and/or data described with regard to the server 700 is present, or otherwise implemented, in one or more of the health provider computing devices 754 and/or a user computing device 756.

The communication bus 710 optionally includes circuitry (sometimes called a chipset) that interconnects and controls communications between system components.

Typically, a healthcare provider tests a patient for various risk factors indicative of particular diseases or conditions. The risk prediction module 722 determines a risk profile 745 (e.g., profile 30 in FIG. 1) for the patient based on the patient's data 746 and/or 748, and in some embodiments, also based on general health data 742. In some embodiments, the patient's data 746/748, as well as the risk profile 745, is stored in the memory 702 as a proprietary dataset of meta-proteomics. In some embodiments, this dataset is mined (e.g., by machine learning module 726) to determine both current and subsequent generations of correlations (e.g., risk models 728). The continuously updated and personalized datasets and risk models make the patient's personal test results (e.g., risk profile 745) increasingly sensitive and accurate. In some embodiments, the system 700 provides each patient with an ever-evolving, ever-improving custom multi-cancer annual screening test, starting in patients' low-risk years, and helping the patients to manage their high-risk years.

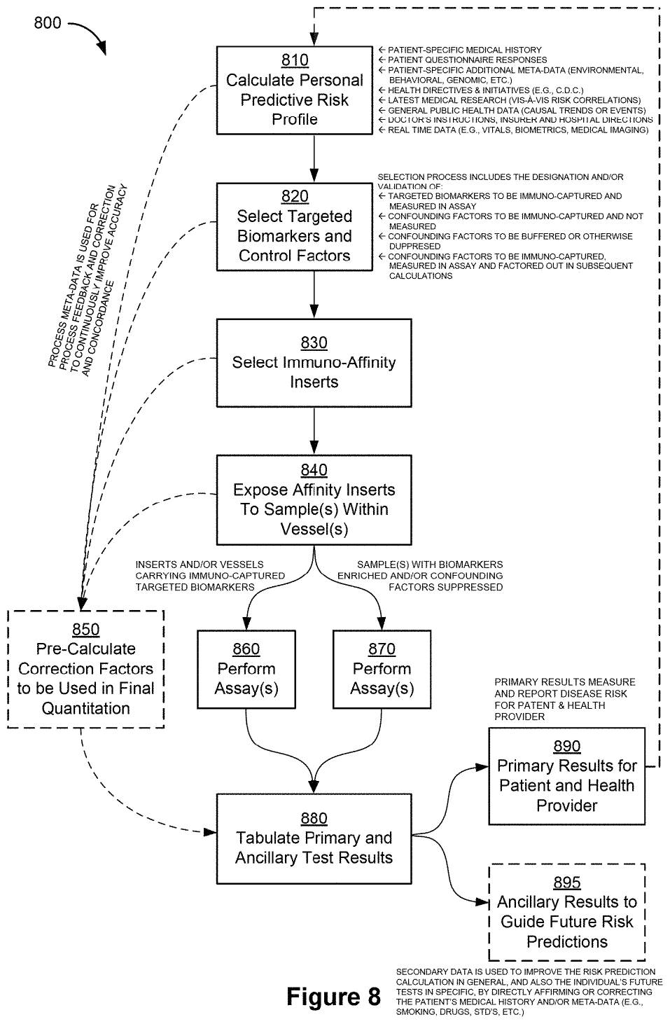

FIG. 8 is a flow diagram illustrating a method 800 of predictive risk calculation in accordance with some embodiments. The method is performed at a server 700, a healthcare provider computing device 754, and/or a user device 756. For example, instructions for performing the method are stored in the memory 702 and executed by the processor(s) 704 of the server 700. In some embodiments, part or all of the instructions for performing the method are stored in memory and executed by processor(s) of the provider device 754 and/or the user device 756.

In step 810, a personal predictive risk profile of a subject is calculated (e.g., by calculation module 724, based on general health data 742 and/or patient data 746 and/or 748). For example, the calculation may be based on any of patient specific medical history, patient questionnaire responses, patient specific additional meta data (e.g., environmental, behavioral, genomic), health directives and initiatives (e.g., CDC directives/initiatives), latest medical research (e.g., pertaining to risk correlations), general public health data (e.g., causal trends or events), doctor's instructions, insurer directives, hospital directives, real-time data (e.g., patient vitals, biometrics, medical imaging).

Targeted biomarkers and control factors are selected in step 820 (e.g., by assay setup module 734). For example, the selection may include the designation and/or validation of targeted biomarkers to be immuno-captured and measured in assay, confounding factors to be immuno-captured and not measured, confounding factors to be buffered or otherwise suppressed, confounding factors to be immuno-captured, measured in assay and factored out in subsequent calculations.

In step 830, immuno-affinity inserts are selected (e.g., by selection module 740). For example, which types of immuno-affinity inserts, how many of each type of immuno-affinity inserts and/or what order the immuno-affinity inserts are to be disposed or used may be selected. The selected immuno-affinity inserts are exposed to sample(s) within vessel(s) or container(s) in step 840. For example, the selected immuno-affinity inserts may be inserted into a vial or tube containing a subject's blood so that the immuno-affinity inserts are in contact with the blood.

In step 850, correction factors to be used in final quantitation are pre-calculated. For example, process metadata may be used for process feedback and correction to continuously improve accuracy and concordance. In some embodiments, calculation module 724 determines one or more probabilities indicative of risk in the context of particular diseases or conditions related to the patient (or type of patient). For example, risk prediction module 722 uses a risk model 728 to predict the results of a particular cancer test for the patient, based on the data calculated, selected, or otherwise obtained, in steps 810 through 840.

In step 860, assay(s) are performed on immuno-affinity inserts and/or vessels carrying immuno-captured targeted biomarkers. In step 870, assay(s) are performed on samples with biomarkers enriched and/or confounding factors suppressed.

In step 880, primary and ancillary test results are tabulated. Primary results are used to measure and report disease risk for a patient in step 890. For example, the disease risk may be reported to the patient and/or a health provider. In step 895, ancillary results may be used to further guide future predictive risk calculations. For example, secondary data is used to improve the risk prediction calculation in general and also to improve the individual's future tests more specifically. This may be done by directly affirming or correcting the patient's medical history and or meta-data (e.g., smoking, drug use, diseases).

In some embodiments, the machine learning module 726 updates the risk model 728 based on the primary results 890 (e.g., results from assays 860 and/or 870) and/or the ancillary results 895. In some embodiments, the machine learning module 726 updates the risk model 728 by associating the results (e.g., 890 and/or 895) with the health data of the subject (e.g., profile data 744), and training the risk model 728 using the health data as input data and the results data as an output label for the input data. In some embodiments, machine learning module 726 uses supervised training, unsupervised training, and/or adversarial training to associate subsequently stored profile data 744 (e.g., from a subsequent doctor/patient visit or test) with the results data if the subsequent health data 744 is similar (e.g., to a predetermined threshold degree of similarity) to the previous health data 744 (e.g., from a previous doctor/patient visit or test). In some embodiments, associating the subsequent health data with the results data includes storing an indication of predicted presence or lack of presence of a particular target biomarker in relation to the subsequent health data.

Method 800 may provide that each test builds a proprietary dataset of meta-proteomics, which is then mined to find the next generation of correlations and to make the individual patient's personal test (e.g., cancer test) more sensitive and accurate. For example, each individual person may have a continuously evolving and improving custom multi-cancer annual screening test starting in the patient's low risk years and helping the patient to manage high risk years. Thus, the disclosed testing may provide for a person to more confidently utilize anti-aging therapies that are undesirable due to increased cancer risks.

The above-described personal proteomic test may be analogized to a search engine that indexes the internet, indexes the individual user's personal preferences and behaviors while using the internet, and utilizes custom (e.g., proprietary) predictive methods to predict what the user will want, what the user will search, what the user will click through and what the user will do with the resulting content.

Any or all aspects discussed above or below may result in a prediction that provides higher accuracy, increased sensitivity, improved precision and better concordance.

Some aspects of the disclosure provide for a multi-factor stage zero cancer screening. Here, the disclosed methodologies provide for early detection of a cancer, that if present at all, is most likely asymptomatic. In some embodiments, the biomarkers may be characteristically rare (e.g., present in the blood in a low amount) or in a particular biological state (e.g., phosphorylated, methylated, misfolded, entangled or aggregated, etc.) including, for example, its in-vivo state (e.g., from raw blood during blood collection).

The above-described immuno-affinity insert may have performance characteristics due to its surface area and/or the number and/or density of affinity molecules bound to its surfaces. Thus, the disclosed immuno-affinity inserts may be designed to capture as many of the targeted biomarkers as possible within an optimal capture capacity of the immuno-affinity insert. In a preferred embodiment, the immuno-affinity insert is neither saturated by the targeted biomarkers nor under-utilized by the capture of an insufficient number of the targeted biomarkers. Without wishing to be bound by a theory of the disclosure, saturated immuno-affinity inserts may be less accurate as they are “capacity limited” and may under-indicate the level of the targeted biomarker within the sample. Similarly, under-utilized immuno-affinity inserts may represent a missed opportunity for using an alternative immuno-affinity insert in its place to capture a different targeted biomarker or confounding constituents in the bodily fluid (e.g., albumen, proteases, hemoglobin or other non-targeted molecular constituents).

Capturing a targeted biomarker within an optimal capture capacity of the immuno-affinity insert may be achieved by anticipating a level of each targeted biomarker and/or confounding constituent present within a biological fluid. In this manner, an appropriate number and type of immuno-affinity insert(s) can be selected, organized, combined and/or utilized in the disclosed methods. Without wishing to be bound by a theory of the disclosure, it is believed that the prediction of the level of a targeted biomarker and/or the level of a confounding constituent may lead to increased accuracy, sensitivity, precision and concordance.

In an embodiment, the disclosed methods may utilize machine learning, combined with scientific health data (e.g., public and private), patient-specific data, occupational data, travel data, mobile device location tracking data, and/or other meta-data to determine an appropriate number and type of immuno-affinity insert(s) to employed in the disclosed methodologies for the capture of a targeted biomarker and/or confounding constituent.

Some aspects of the disclosure provide for recurring screening (e.g., annual screening) with a feedback loop based upon previously measured target biomarkers and target biomarker enrichment meta-data. For example, a target biomarker captured in a first cancer screening may guide the selection of an appropriate number and type of immuno-affinity insert for subsequent cancer screenings (e.g., once a month, once a year, or once every other year, etc.). In this manner, patient specific meta-data obtained from a first screening may be used to guide the selection of an appropriate number and type of immuno-affinity insert used for a second cancer screening and meta-data obtained from the second screening may be used to guide the selection of an appropriate number and type of immuno-affinity insert used for a third screening, and so on. For example, where the first screening identifies that the subject has (or is at risk of developing) a certain cancer, a subsequent (e.g., second) screening may include an appropriate number and type of immuno-affinity insert specific for that particular cancer and other diseases or disorders that are characteristic of a subject having the specific cancer. Further, meta-data concerning diseases and/or disorders not anticipated to be present in a subject due to a biomarker (or a pattern of biomarkers) identified in a first screening may be used to guide an appropriate number and type of immuno-affinity insert specific for certain diseases and/or disorders in a second screening. Alternatively, the subsequent (e.g., second) screening may include an appropriate number and type of immuno-affinity insert specific for cancers other than the cancer(s), and optionally the diseases and/or disorders, identified by the first screening.

For example, a biomarker for body mass index may be used to estimate subsequent shifts in a subject's cancer risk profile such as a woman's peri-menopausal increase heart disease risk. As another example, levels of free testosterone may be used to estimate future prostate cancer risk. In yet another example, molecular biomarkers for smoking, drinking or drug history may serve to confirm, contradict or contextualize the information given by a patient about their health history. Each of these biomarkers may be used to guide the selection of an appropriate number and type of immuno-affinity insert.

The number of immune-affinity inserts used in the methods disclosed herein (including the nature of the affinity molecules on those immuno-affinity inserts) and/or the volume of fluid into which the immuno-affinity inserts are inserted may introduce a measurement bias. Thus, in some embodiments, the devices disclosed herein may include an immuno-affinity insert having affinity molecules specific for a control biomarker. In this manner, the amount of the control biomarker present in a known volume of bodily fluid such as blood may be used to determine the amount of control biomarker in the tested bodily fluid and adjust for any measurement bias. For example, where a control biomarker A is known to be present at a concentration of 10 ng/mL in human blood, then the capture of 100 ng of biomarker A by the devices disclosed herein suggests that the initial volume of tested blood was 10 mL. The determination of the initial volume of bodily fluid may be used to assess whether the number of affinity inserts was insufficient, sufficient, or overly sufficient for a target biomarker based on the known concentration of the target biomarker per unit (e.g., mL) of bodily fluid.

In one or more embodiments, the amount of a target biomarker bound to affinity molecules specific for the target biomarker may be adjusted to account for unspecific or background binding to the affinity molecules by other biomarkers or contaminants present in the bodily fluid. For example, the amount of a target biomarker bound to an affinity molecule may be adjusted by subtracting away an expected or known background level of nonspecific binding or contaminant binding to the affinity molecules.

In one or more embodiments, methods for enriching a target biomarker from a bodily fluid of a subject is provided. The methods include predicting that a target biomarker is present in the bodily fluid; contacting the bodily fluid with one or more immuno-affinity inserts coated with affinity molecules specific for the target biomarker under conditions for the affinity molecules to bind the target biomarker; and separating the affinity molecules bound to the target biomarker from the bodily fluid.

In some embodiments of each or any of the above- or below-mentioned embodiments, the methods further comprise purifying the target biomarker from the affinity molecules.

In some embodiments of each or any of the above- or below-mentioned embodiments, the methods further comprise purifying the target biomarker from the affinity molecules.

In some embodiments of each or any of the above- or below-mentioned embodiments, the methods further comprise detecting the target biomarker.

In some embodiments of each or any of the above- or below-mentioned embodiments, the target biomarker is predicted to be present in the bodily fluid based on the risk profile for the subject.

In some embodiments of each or any of the above- or below-mentioned embodiments, the subject's risk profile is based on the subject's age, weight, height, ethnicity, medical history, a physical examination of the subject, an interview with the subject, a computerized test result obtained from the subject, a medical image obtained from the subject, a risk-related public health initiative or directive, scientific health data (e.g., public and private), subject-specific data, occupational data specific for the subject, travel data specific to the subject, the subject's mobile device location tracking data, and/or other meta-data or any combination thereof.

In some embodiments of each or any of the above- or below-mentioned embodiments, the subject's risk profile is determined based upon the subject's membership in a class of subjects, wherein all subjects in the class are at risk of a common disease, disorder, or condition.

In some embodiments of each or any of the above- or below-mentioned embodiments, the target biomarker is a protein, a nucleic acid, a peptide, a polymer, an amino acid or a glycan.

In some embodiments of each or any of the above- or below-mentioned embodiments, the bodily fluid is blood, plasma, saliva, tears, urine, amniotic fluid, cerebrospinal fluid, pleural fluid, or peritoneal fluid.

In some embodiments of each or any of the above- or below-mentioned embodiments, the bodily fluid is blood.

In some embodiments of each or any of the above- or below-mentioned embodiments, the immuno-affinity inserts are magnetic.

In some embodiments of each or any of the above- or below-mentioned embodiments, the immuno-affinity inserts are fluorescent.

In some embodiments of each or any of the above- or below-mentioned embodiments, the bodily fluid is contacted with more than one set of immuno-affinity inserts, and wherein each set is coated with a different affinity molecule.

In some embodiments of each or any of the above- or below-mentioned embodiments, the subject is predicted to have a cancer.

In some embodiments of each or any of the above- or below-mentioned embodiments, the cancer is non-melanoma skin cancer, lung cancer, breast cancer, prostate cancer, colorectal cancer, bladder cancer, melanoma, non-Hodgkin lymphoma, ovarian cancer, cervical cancer, or pancreatic cancer.

In some embodiments of each or any of the above- or below-mentioned embodiments, the subject is predicted to have Alzheimer's disease.

In some embodiments of each or any of the above- or below-mentioned embodiments, the target biomarker is tau protein.

In some embodiments of each or any of the above- or below-mentioned embodiments, the tau protein is hyperphosphorylated.

In some embodiments of each or any of the above- or below-mentioned embodiments, the affinity molecules are present in a soluble affinity matrix (SAM).

In some embodiments of each or any of the above- or below-mentioned embodiments, release of immuno-affinity inserts from the SAM is triggered by pH or osmolality of the bodily fluid.

In some embodiments of each or any of the above- or below-mentioned embodiments, a first set of immuno-affinity inserts is present in a first layer of the SAM.

In some embodiments of each or any of the above- or below-mentioned embodiments, a second set of immuno-affinity inserts is present in a second layer of the SAM.

In some embodiments of each or any of the above- or below-mentioned embodiments, the SAM comprises a first layer that elutes a set of immuno-affinity inserts having affinity molecules that specifically bind one or more undesired biomarkers.

In some embodiments of each or any of the above- or below-mentioned embodiments, the SAM comprises a second layer that elutes a set of immuno-affinity inserts coated with affinity molecules specific for a first target biomarker.

In some embodiments of each or any of the above- or below-mentioned embodiments, the SAM comprises a third layer that elutes a set of immuno-affinity inserts coated with affinity molecules specific for a second target biomarker.

In some embodiments of each or any of the above- or below-mentioned embodiments, a first set of immuno-affinity inserts is released from the SAM at a first predetermined time and a second set of immuno-affinity inserts is released from the SAM at a second predetermined time.

In some embodiments of each or any of the above- or below-mentioned embodiments, the second predetermined time is later than the first predetermined time.

In some embodiments of each or any of the above- or below-mentioned embodiments, the affinity molecules are selected from the group consisting of antibodies, antibody fragments, enzymes, fragments of enzymes, enzyme substrates, fragments of enzyme substrates, nucleotides, oligonucleotides, polynucleotides, receptors, aptamers, nanobodies, fragments of receptors, ligands, fragments of enzymes, other proteins, amino acids, peptides, polypeptides, oligopeptides, saccharides, disaccharides, polysaccharides, glycoproteins, proteoglycans, and natural and synthetic polymers.

In some embodiments of each or any of the above- or below-mentioned embodiments, the immuno-affinity inserts comprise one of particles, tubes and filaments.

The present disclosure also provides methods for enriching a target biomarker from a bodily fluid of a subject comprising obtaining the bodily fluid comprising a target biomarker from the subject; flowing the bodily fluid through a first component containing one or more sets of immuno-affinity inserts coated with affinity molecules specific for the target biomarker, and flowing the bodily fluid through a second component that retain the immuno-affinity inserts bound to the target biomarker.

In some embodiments of each or any of the above- or below-mentioned embodiments, the set of immuno-affinity inserts are magnetic or fluorescent.

In some embodiments of each or any of the above- or below-mentioned embodiments, the first component is a cartridge.

In some embodiments of each or any of the above- or below-mentioned embodiments, the second component is a vial or a cartridge.

In some embodiments of each or any of the above- or below-mentioned embodiments, the target biomarker is associated with a disease, disorder, or condition.

In some embodiments of each or any of the above- or below-mentioned embodiments, the target biomarker is tau protein and the disease is Alzheimer's disease.

In some embodiments of each or any of the above- or below-mentioned embodiments, the method further comprise purifying the target biomarker from the affinity molecules.

In some embodiments of each or any of the above- or below-mentioned embodiments, the methods further comprise detecting the target biomarker.

In some embodiments of each or any of the above- or below-mentioned embodiments, the target biomarker is predicted to be present in the bodily fluid based on the subject's risk profile.

In some embodiments of each or any of the above- or below-mentioned embodiments, the subject's risk profile is based on the subject's age, weight, height, ethnicity, medical history, a physical examination of the subject, an interview with the subject, a computerized test result obtained from the subject, a medical image obtained from the subject, a risk-related public health initiative or directive, scientific health data (e.g., public and private), subject-specific data, occupational data specific for the subject, travel data specific to the subject, the subject's mobile device location tracking data, and/or other meta-data or any combination thereof. In some embodiments, the subject's risk profile is based on the results obtained for enriching (or screening for the presence or absence of) a target biomarker using the device and/or methods disclosed herein.

In some embodiments of each or any of the above- or below-mentioned embodiments, the subject's risk profile is determined based upon the subject's membership in a class of subjects, wherein all subjects in the class are at risk of a common disease, disorder, or condition.

In some embodiments of each or any of the above- or below-mentioned embodiments, the affinity molecules for a disease, disorder, or condition are selected for use in the device where the subject's risk profile indicates that the likelihood of developing the disease, disorder, or condition is 50% or greater, 55% or greater, 60% or greater, 65% or greater, 70% or greater, 75% or greater, 80% or greater, 85% or greater, 90% or greater, or 95% or greater.

In some embodiments of each or any of the above- or below-mentioned embodiments, the target biomarker is a protein, a nucleic acid, a peptide, a polymer, an amino acid or a glycan.

In some embodiments of each or any of the above- or below-mentioned embodiments, the bodily fluid is blood, plasma, saliva, tears, urine, amniotic fluid, cerebrospinal fluid, pleural fluid, or peritoneal fluid.

In some embodiments of each or any of the above- or below-mentioned embodiments, the bodily fluid is blood.

In some embodiments of each or any of the above- or below-mentioned embodiments, the immuno-affinity inserts comprise one of particles, tubes and filaments.

In some embodiments of each or any of the above- or below-mentioned embodiments, the bodily fluid is contacted with more than one set of immuno-affinity inserts, and wherein each set is coated with a different affinity molecule.

In some embodiments of each or any of the above- or below-mentioned embodiments, the subject is predicted to have a cancer.

In some embodiments of each or any of the above- or below-mentioned embodiments, the cancer is non-melanoma skin cancer, lung cancer, breast cancer, prostate cancer, colorectal cancer, bladder cancer, melanoma, non-Hodgkin lymphoma, ovarian cancer, cervical cancer, or pancreatic cancer.

In some embodiments of each or any of the above- or below-mentioned embodiments, the subject is predicted to have Alzheimer's disease.

In some embodiments of each or any of the above- or below-mentioned embodiments, the target biomarker is tau protein.

In some embodiments of each or any of the above- or below-mentioned embodiments, the tau protein is hyperphosphorylated.

In some embodiments of each or any of the above- or below-mentioned embodiments, the affinity molecules are present in a soluble affinity matrix (SAM).

In some embodiments of each or any of the above- or below-mentioned embodiments, release of the set of immuno-affinity inserts from the SAM is triggered by wetness, pH or osmolality of the bodily fluid.

In some embodiments of each or any of the above- or below-mentioned embodiments, a first set of immuno-affinity inserts is present in a first layer of the SAM.

In some embodiments of each or any of the above- or below-mentioned embodiments, a second set of immuno-affinity inserts is present in a second layer of the SAM.

In some embodiments of each or any of the above- or below-mentioned embodiments, the SAM comprises a first layer that elutes a set of immuno-affinity inserts having affinity molecules that specifically bind one or more undesired biomarkers.

In some embodiments of each or any of the above- or below-mentioned embodiments, the SAM comprises a second layer that elutes the set of immuno-affinity inserts coated with affinity molecules specific for the target biomarker.

In some embodiments of each or any of the above- or below-mentioned embodiments, the first set of immuno-affinity inserts is released from the SAM at a first predetermined time and the second set of immuno-affinity inserts is released from the SAM at a second predetermined time.

In some embodiments of each or any of the above- or below-mentioned embodiments, the second predetermined time is later than the first predetermined time.

In some embodiments of each or any of the above- or below-mentioned embodiments, the affinity molecules are selected from the group consisting of antibodies, antibody fragments, enzymes, fragments of enzymes, enzyme substrates, fragments of enzyme substrates, nucleotides, oligonucleotides, polynucleotides, receptors, aptamers, nanobodies, fragments of receptors, ligands, fragments of enzymes, other proteins, amino acids, peptides, polypeptides, oligopeptides, saccharides, disaccharides, polysaccharides, glycoproteins, proteoglycans, and natural and synthetic polymers.

The present disclosure also provides methods for diagnosing a subject with a disease, disorder, or condition comprising predicting that a target biomarker is present in the bodily fluid; obtaining a bodily fluid from the subject; flowing the bodily fluid through a device containing affinity molecules specific for the target biomarker; and identifying the presence of the target biomarker in the bodily fluid, wherein the subject is diagnosed with the disease, disorder, or condition where the target biomarker is identified in the bodily fluid.

In some embodiments of each or any of the above- or below-mentioned embodiments, the methods further comprise purifying the target biomarker from the affinity molecules.

In some embodiments of each or any of the above- or below-mentioned embodiments, the methods further comprise detecting the target biomarker.

In some embodiments of each or any of the above- or below-mentioned embodiments, the target biomarker is predicted to be present in the bodily fluid based on the subject's risk profile.

In some embodiments of each or any of the above- or below-mentioned embodiments, the subject's risk profile is based on the subject's age, weight, height, ethnicity, medical history, a physical examination of the subject, an interview with the subject, a computerized test result obtained from the subject, a medical image obtained from the subject, a risk-related public health initiative or directive, scientific health data (e.g., public and private), subject-specific data, occupational data specific for the subject, travel data specific to the subject, the subject's mobile device location tracking data, and/or other meta-data or any combination thereof. In some embodiments, the subject's risk profile is based on the results obtained for enriching (or screening for the presence or absence of) a target biomarker using the device and/or methods disclosed herein.

In some embodiments of each or any of the above- or below-mentioned embodiments, the subject's risk profile is determined based upon the subject's membership in a class of subjects, wherein all subjects in the class are at risk of a common disease, disorder, or condition.

In some embodiments of each or any of the above- or below-mentioned embodiments, the target biomarker is a protein, a nucleic acid, a peptide, a polymer, an amino acid or a glycan.

In some embodiments of each or any of the above- or below-mentioned embodiments, the bodily fluid is blood, plasma, saliva, tears, urine, amniotic fluid, cerebrospinal fluid, pleural fluid, or peritoneal fluid.

In some embodiments of each or any of the above- or below-mentioned embodiments, the bodily fluid is blood.

In some embodiments of each or any of the above- or below-mentioned embodiments, the immuno-affinity inserts are magnetic.

In some embodiments of each or any of the above- or below-mentioned embodiments, the immuno-affinity inserts are fluorescent.

In some embodiments of each or any of the above- or below-mentioned embodiments, the immuno-affinity inserts comprise one of particles, tubes and filaments.

In some embodiments of each or any of the above- or below-mentioned embodiments, the bodily fluid is contacted with at more than one set of immuno-affinity inserts, and wherein each set is coated with a different affinity molecule.

In some embodiments of each or any of the above- or below-mentioned embodiments, the subject is predicted to have a cancer.

In some embodiments of each or any of the above- or below-mentioned embodiments, the cancer is non-melanoma skin cancer, lung cancer, breast cancer, prostate cancer, colorectal cancer, bladder cancer, melanoma, non-Hodgkin lymphoma, ovarian cancer, cervical cancer, or pancreatic cancer.

In some embodiments of each or any of the above- or below-mentioned embodiments, the subject is predicted to have Alzheimer's disease.

In some embodiments of each or any of the above- or below-mentioned embodiments, the target biomarker is tau protein.

In some embodiments of each or any of the above- or below-mentioned embodiments, the tau protein is hyperphosphorylated.

In some embodiments of each or any of the above- or below-mentioned embodiments, the affinity molecules are present in a soluble affinity matrix (SAM).

In some embodiments of each or any of the above- or below-mentioned embodiments, the SAM additionally comprises a preservative that protects a target molecule from degradation or biochemical modification.

In some embodiments of each or any of the above- or below-mentioned embodiments, a release of the set of immuno-affinity inserts from the SAM is triggered by wetness, pH or osmolality of the bodily fluid.

In some embodiments of each or any of the above- or below-mentioned embodiments, a first set of immuno-affinity inserts is present in a first layer of the SAM.

In some embodiments of each or any of the above- or below-mentioned embodiments, a second set of immuno-affinity inserts is present in a second layer of the SAM.

In some embodiments of each or any of the above- or below-mentioned embodiments, the SAM comprises a first layer that elutes a set of immuno-affinity inserts having affinity molecules that specifically bind one or more undesired biomarkers.

In some embodiments of each or any of the above- or below-mentioned embodiments, the SAM comprises a second layer that elutes the set of immuno-affinity inserts coated with affinity molecules specific for the target biomarker.

In some embodiments of each or any of the above- or below-mentioned embodiments, the first set of immuno-affinity inserts is released from the SAM at a first predetermined time and the second set of immuno-affinity inserts is released from the SAM at a second predetermined time.

In some embodiments of each or any of the above- or below-mentioned embodiments, the second predetermined time is later than the first predetermined time.

In some embodiments of each or any of the above- or below-mentioned embodiments, the affinity molecules are selected from the group consisting of antibodies, antibody fragments, enzymes, fragments of enzymes, enzyme substrates, fragments of enzyme substrates, nucleotides, oligonucleotides, polynucleotides, receptors, aptamers, nanobodies, fragments of receptors, ligands, fragments of enzymes, other proteins, amino acids, peptides, polypeptides, oligopeptides, saccharides, disaccharides, polysaccharides, glycoproteins, proteoglycans, and natural and synthetic polymers.

In some embodiments of each or any of the above- or below-mentioned embodiments, the SAM comprises immuno-affinity inserts comprising one of particles, tubes and filaments.

The present disclosure also provides a device for enriching a target biomarker from a bodily fluid of a subject comprising one or more sets of immuno-affinity inserts coated with affinity molecules specific for the target biomarker, wherein the affinity molecules are selected based on a risk profile for the subject.

In some embodiments of each or any of the above- or below-mentioned embodiments, the device further comprises i) a first component containing the one or more sets of immuno-affinity inserts and ii) a second component that retains immuno-affinity inserts bound to the target biomarker, wherein the first component is in fluid connection to the second component.

In some embodiments of each or any of the above- or below-mentioned embodiments, the first component is a cartridge.

In some embodiments of each or any of the above- or below-mentioned embodiments, the cartridge is selected by a computer controlled machine.

In some embodiments of each or any of the above- or below-mentioned embodiments, the cartridge is preloaded with the one or more sets of immuno-affinity inserts coated with affinity molecules specific for the target biomarker by a computer controlled machine.

In some embodiments of each or any of the above- or below-mentioned embodiments, the immuno-affinity inserts are magnetic.

In some embodiments of each or any of the above- or below-mentioned embodiments, the second component contains a magnetic element to capture the immuno-affinity inserts.

In some embodiments of each or any of the above- or below-mentioned embodiments, the magnetic element is reusable.

In some embodiments of each or any of the above- or below-mentioned embodiments, the magnetic element is located on the outside surface of the second component.

In some embodiments of each or any of the above- or below-mentioned embodiments, the affinity molecules are selected from the group consisting of antibodies, antibody fragments, enzymes, fragments of enzymes, enzyme substrates, fragments of enzyme substrates, nucleotides, oligonucleotides, polynucleotides, receptors, aptamers, nanobodies, fragments of receptors, ligands, fragments of enzymes, other proteins, amino acids, peptides, polypeptides, oligopeptides, saccharides, disaccharides, polysaccharides, glycoproteins, proteoglycans, and natural and synthetic polymers.

In some embodiments of each or any of the above- or below-mentioned embodiments, there are at least two sets of paramagnetic immuno-affinity inserts, wherein each set of paramagnetic immuno-affinity inserts is coated with a different affinity molecule, and wherein each set of paramagnetic immuno-affinity inserts is marked with a different fluorescent dye or combination of fluorescent dyes.

In some embodiments of each or any of the above- or below-mentioned embodiments, the paramagnetic immuno-affinity inserts are continually released from the first component as the bodily fluid flows through the first component.

In some embodiments of each or any of the above- or below-mentioned embodiments, the target biomarker is predicted to be present in the bodily fluid based on the subject's risk profile.

In some embodiments of each or any of the above- or below-mentioned embodiments, the subject's risk profile is based on the subject's age, weight, height, ethnicity, medical history, a physical examination of the subject, an interview with the subject, a computerized test result obtained from the subject, a medical image obtained from the subject, a risk-related public health initiative or directive, scientific health data (e.g., public and private), subject-specific data, occupational data specific for the subject, travel data specific to the subject, the subject's mobile device location tracking data, and/or other meta-data or any combination thereof. In some embodiments, the subject's risk profile is based on the results obtained for enriching (or screening for the presence or absence of) a target biomarker using the device and/or methods disclosed herein.

In some embodiments of each or any of the above- or below-mentioned embodiments, the subject's risk profile is determined based upon the subject's membership in a class of subjects, wherein all subjects in the class are at risk of a common disease, disorder, or condition.

In some embodiments of each or any of the above- or below-mentioned embodiments, the device further comprises a computer controlled machine that selects immuno-affinity inserts.

In some embodiments of each or any of the above- or below-mentioned embodiments, the immuno-affinity inserts are filaments.

In some embodiments of each or any of the above- or below-mentioned embodiments, the filaments are coiled, compound-coiled, straight or matted.

In some embodiments of each or any of the above- or below-mentioned embodiments, the filaments are a fiber, a micro-tubule or a nanotube.

In some embodiments of each or any of the above- or below-mentioned embodiments, the device further comprises a filament structure having a plurality of filaments attached together in a horsetail configuration.

In some embodiments of each or any of the above- or below-mentioned embodiments, the device further comprises a filament structure having a plurality of filaments bound or wound to a central support structure.

The present disclosure also provides computer-implemented methods, comprising, at a server system including one or more processors and memory storing one or more programs for execution by the one or more processors: predicting, based on a predictive risk model, that a target biomarker is present in bodily fluid of a first subject; receiving first assay data including identification of presence or lack of presence of the target biomarker; and updating the predictive risk model, based on the first assay data.

In some embodiments of each or any of the above- or below-mentioned embodiments, the predictive risk model is based on stored health data of the first subject including one or more of: medical history data, environmental data, behavioral data, genomic data, vitals data, biometrics data, and/or a medical image of the first subject.

In some embodiments of each or any of the above- or below-mentioned embodiments, the predictive risk model is based on stored health data of a plurality of subjects including one or more of: medical history data, environmental data, behavioral data, genomic data, vitals data, biometrics data, and/or medical images of the plurality of subjects.

In some embodiments of each or any of the above- or below-mentioned embodiments, the predictive risk model is further based on general public health data, including one or more of: a health directive or initiative, medical research, and/or health data based on causal trends or events.

In some embodiments of each or any of the above- or below-mentioned embodiments, updating the predictive risk model includes: associating the first assay data with the stored health data of the first subject; and training the predictive risk model using the stored health data of the first subject as input data and the first assay data as an output label for the input data.

In some embodiments of each or any of the above- or below-mentioned embodiments, training the predictive risk model includes using supervised training, unsupervised training, and/or adversarial training to associate subsequently stored health data with the first assay data if the subsequently stored health data is similar to the previously stored health data.

In some embodiments of each or any of the above- or below-mentioned embodiments, associating the subsequently stored health data with the first assay data includes storing an indication of predicted presence or lack of presence of the target biomarker in relation to the subsequently stored health data.

In some embodiments of each or any of the above- or below-mentioned embodiments, the methods further comprise predicting, based on the updated predictive risk model, that the target biomarker is present in bodily fluid of a second subject.

In some embodiments of each or any of the above- or below-mentioned embodiments, the methods further comprise predicting, based on the updated predictive risk model, that the target biomarker is present in a subsequently obtained sample of bodily fluid of the first subject.

In some embodiments of each or any of the above- or below-mentioned embodiments, the methods further comprise predicting, based on the updated predictive risk model, that the target biomarker is no longer present in bodily fluid of the first subject.

Features of the embodiments described herein can be implemented in, using, or with the assistance of a computer program product, such as a storage medium (media) or computer readable storage medium (media) having instructions stored thereon/in which can be used to program a processing system to perform any of the features presented herein. The storage medium (e.g., the memory 102 and the memory 202) can include, but is not limited to, high-speed random access memory, such as DRAM, SRAM, DDR RAM or other random access solid state memory devices, and may include non-volatile memory, such as one or more magnetic disk storage devices, optical disk storage devices, flash memory devices, or other non-volatile solid state storage devices. In some embodiments, the memory 102 and the memory 202 include one or more storage devices remotely located from the CPU(s) 104 and 204. The memory 102 and the memory 202, or alternatively the non-volatile memory device(s) within these memories, comprises a non-transitory computer readable storage medium.

Communication systems as referred to herein (e.g., the communication system 108 and the communication system 208) optionally communicate via wired and/or wireless communication connections. Communication systems optionally communicate with networks (e.g., the networks 150 and 152), such as the Internet, also referred to as the World Wide Web (WWW), an intranet and/or a wireless network, such as a cellular telephone network, a wireless local area network (LAN) and/or a metropolitan area network (MAN), and other devices by wireless communication. Wireless communication connections optionally use any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 102.11a, IEEE 102.11ac, IEEE 102.11ax, IEEE 102.11b, IEEE 102.11g and/or IEEE 102.11n), voice over Internet Protocol (VoIP), Wi-MAX, a protocol for e mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

Although the disclosure has been described and illustrated with a certain degree of particularity, it is understood that the present disclosure has been made only by way of example, and that numerous changes in the combination and arrangement of parts can be resorted to by those skilled in the art without departing from the scope of the disclosure, as hereinafter claimed.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

Thus, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. Method claims may be provided to present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary and Brief Description of the Drawings are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in any claim. Rather, as the following claims reflect, inventive subject matter lies in less than all features of a single disclosed configuration or operation.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A method for enriching a target biomarker from a bodily fluid of a subject, the method comprising:
predicting that a target biomarker is present in the bodily fluid;
contacting the bodily fluid with one or more immuno-affinity inserts coated with affinity molecules specific for the target biomarker under conditions for the affinity molecules to bind the target biomarker; and
separating the affinity molecules bound to the target biomarker from the bodily fluid.

2. The method of claim 1 further comprising purifying the target biomarker from the affinity molecules.

3. The method of claim 1 further comprising detecting the target biomarker.

4. The method of claim 1, wherein the target biomarker is predicted to be present in the bodily fluid based on a risk profile for the subject.

5. The method of claim 4, wherein the subject's risk profile is based on age, weight, height, ethnicity, medical history, a physical examination, a patient interview, a computerized test result, a medical image, a risk-related public health initiative or directive, or any combination thereof.

6. The method of claim 4, wherein the subject's risk profile is determined based upon the subject's membership in a class of subjects, wherein all subjects in the class are at risk of a common disease, disorder, or condition.

7. The method of claim 1, wherein the target biomarker is a protein, a nucleic acid, a peptide, a polymer, an amino acid or a glycan.

8. The method of claim 1, wherein the bodily fluid is blood, plasma, saliva, tears, urine, amniotic fluid, cerebrospinal fluid, pleural fluid, or peritoneal fluid.

9. The method of claim 8, wherein the bodily fluid is blood.

10.-17. (canceled)

18. The method of claim 1, wherein the affinity molecules are present in a soluble affinity matrix (SAM).

19.-26. (canceled)

27. The method of claim 1, wherein the affinity molecules are selected from the group consisting of antibodies, antibody fragments, enzymes, fragments of enzymes, enzyme substrates, fragments of enzyme substrates, nucleotides, oligonucleotides, polynucleotides, receptors, aptamers, nanobodies, fragments of receptors, ligands, fragments of enzymes, other proteins, amino acids, peptides, polypeptides, oligopeptides, saccharides, disaccharides, polysaccharides, glycoproteins, proteoglycans, and natural and synthetic polymers.

28. (canceled)

29. A method for enriching a target biomarker from a bodily fluid of a subject, the method comprising:
a) obtaining the bodily fluid comprising a target biomarker from the subject;
b) flowing the bodily fluid through a first component containing one or more sets of immuno-affinity inserts coated with affinity molecules specific for the target biomarker; and
c) flowing the bodily fluid through a second component that retains immuno-affinity inserts bound to the target biomarker.

30. The method of claim 29, wherein the immuno-affinity inserts are magnetic or fluorescent.

31. The method of claim 29, wherein the first component is a cartridge.

32. The method of claim 29, wherein the second component is a vial or a cartridge.

33. The method of claim 29, wherein the target biomarker is associated with a disease, disorder, or condition.

34. The method of claim 33, wherein the target biomarker is tau protein and the disease is Alzheimer's disease.

35.-59. (canceled)

60. A method for diagnosing a subject with a disease, disorder, or condition, the method comprising:
a) predicting that a target biomarker is present in a bodily fluid;
b) obtaining a bodily fluid from the subject;
c) flowing the bodily fluid through a device containing affinity molecules specific for the target biomarker; and
d) identifying the presence of the target biomarker in the bodily fluid,
wherein the subject is diagnosed with the disease, disorder, or condition where the target biomarker is identified in the bodily fluid.

61. The method of claim 60 further comprising purifying the target biomarker from the affinity molecules.

62. The method of claim 60 further comprising detecting the target biomarker.

63. The method of claim 60, wherein the target biomarker is predicted to be present in the bodily fluid based on a risk profile for the subject.

64. The method of claim 63, wherein the subject's risk profile is based on age, weight, height, ethnicity, medical history, a physical examination, a patient interview, a computerized test result, a medical image, a risk-related public health initiative or directive, or any combination thereof.

65. The method of claim 63, wherein the subject's risk profile is determined based upon the subject's membership in a class of subjects, wherein all subjects in the class are at risk of a common disease, disorder, or condition.

66. The method of claim 60, wherein the target biomarker is a protein, a nucleic acid, a peptide, a polymer, an amino acid or a glycan.

67.-88. (canceled)

89. A device for enriching a target biomarker from a bodily fluid of a subject, the device comprising:
one or more sets of immuno-affinity inserts coated with affinity molecules specific for the target biomarker, wherein the affinity molecules are selected based on a risk profile for the subject.

90. The device of claim 89 further comprising i) a first component containing the one or more sets of immuno-affinity inserts and ii) a second component that retains immuno-affinity inserts bound to the target biomarker, wherein the first component is in fluid connection to the second component.

91. The device of claim 90, wherein the first component is a cartridge.

92.-110. (canceled)

111. A computer-implemented method, comprising:
at a server system including one or more processors and memory storing one or more programs for execution by the one or more processors:
predicting, based on a predictive risk model, that a target biomarker is present in bodily fluid of a first subject;
receiving first assay data including identification of presence or lack of presence of the target biomarker; and
updating the predictive risk model, based on the first assay data.

112.-120. (canceled)
